(19) United States
(12) Patent Application Publication
(10) Pub. No.: US 2010/0028460 A1
(43) Pub. Date: Feb. 4, 2010

(54) IMPROVEMENTS IN RELATION TO CANCER THERAPY

(76) Inventors: Roland Wolf, Dundee (GB); John Hayes, Dundee (GB); Xiu Jun Wang, Dundee (GB)

Correspondence Address: GIFFORD, KRASS, SPRINKLE,ANDERSON & CITKOWSKI, P.C, PO BOX 7021, TROY, MI, 48007-7021, US

(21) Appl. No.: 12/375,051

(22) PCT Filed: Jul. 25, 2007
(86) PCT No.: PCT/GB07/02833

§ 371 Date: Jan. 26, 2009

(30) Foreign Application Priority Data
Jul. 25, 2006 (GB) 0614707.8
Feb. 2, 2007 (GB) 0702003.5

(51) Int. Cl. A61K 31/07; A61P 35/00; A61P 17/06; C12Q 1/68; C12N 5/071; A61K 33/24; A61K 31/35; A61K 31/704; A61K 35/74

(52) U.S. Cl. 424/649; 435/6; 435/366; 514/725; 514/451; 514/34; 424/780

(57) ABSTRACT

The present invention relates to an improved assay for identifying compounds that may be of use in conjunction with cancer chemotherapeutic agents and anti-proliferative agents, to improve efficacy of such agents and/or render effective compounds with relatively little therapeutic activity. There is also provided a class of compounds of formula (I) and retinoids identified by said assay which may be used in a combination therapy, with current and novel agents, to treat cancers and other diseases associated with abnormal host cell proliferation, such as psoriasis.

### FIELD OF THE INVENTION

The present invention relates to an improved assay for identifying compounds that may be of use in conjunction with cancer chemotherapeutic agents and/or anti-proliferative agents, to improve efficacy of such agents and/or render effective compounds with relatively little therapeutic activity. There is also provided a class of compounds identified by said assay which may be used in a combination therapy, with current and novel agents, to treat cancers and other diseases associated with abnormal host cell proliferation, such as psoriasis.

### BACKGROUND TO THE INVENTION

### Introduction

Drug-metabolising enzymes such as NAD(P)H:quinone oxidoreductase 1 (NQO1), aldo-keto reductase (AKR) microsomal epoxide hydrolase, UDP-glucuronosyl transferases and glutathione S-transferases (GST), along with reduced glutathione (GSH) and its biosynthetic enzymes, glutamate cysteine ligase (GCL, comprising GCLC and GCLM subunits) and GSH synthase, protect cells against carcinogenic electrophiles as well as reactive oxygen species (ROS) (Hayes & Wolf, 1990; Nioi & Hayes, 2004). This defence can be up-regulated in response to redox stressors, thereby allowing cells to adapt and become resistant to the presence of pro-oxidants and electrophiles. The defence is also overexpressed in certain tumours. Induction of these genes is controlled primarily by Nrf2 (McMahon et al., 2001; Lee et al., 2003), a transcription factor belonging to the family of cap ‘n’ collar (CNC) basic-region leucine zipper (bZIP) proteins (Hayes & McMahon, 2001; Motohashi et al., 2002; Kobayashi et al., 2005). Nrf2 mediates induction of detoxication and antioxidant genes that contain an antioxidant response element (ARE, 5′-(A/G)TGACNNNGC(A/G)-3′) in their promoters (Rushmore et al., 1991; Friling et al., 1992; Nguyen et al., 1994; Wasserman & Fahl, 1997; Sasaki et al., 2002; Mulcahy et al., 1997; Erickson et al., 2002; Ikeda et al., 2002; Kepa et al., 2003; Nioi, et al., 2003; Jowsey et al., 2003); the ARE has also occasionally been referred to in the literature as the electrophile response element (EpRE). A key role for Nrf2 in controlling the ability of cells to withstand harmful environmental agents has been demonstrated by studies in which Nrf2 knockout mice (Itoh et al., 1997) have been shown to exhibit sensitivity to hyperoxia-induced lung injury (Cho et al., 2002), cigarette smoke-induced emphysema, and increased susceptibility to toxic xenobiotics including carcinogens (Aoki et al., 2001; Chan et al., 1999; Enomoto et al., 2001; Iida et al., 2004; Ramos-Gomez et al., 2001).

The activity of Nrf2 is repressed by binding to an inhibitory factor, Kelch-like ECH associated protein 1 (Keap1) that may tether the bZIP protein in the cytoplasm (Itoh et al., 1999; Kang et al., 2004). Alternatively, Keap1 may facilitate degradation of Nrf2 because it acts as a cullin-3 substrate adaptor, and thereby promotes ubiquitylation and proteasomal degradation of the bZIP protein (McMahon et al., 2003; Kobayashi et al., 2004; Cullinan et al., 2004; Zhang et al., 2004; Furukawa et al, 2005). Electrophilic agents and oxidative stressors modify Keap1 and prevent it from targeting Nrf2 for degradation (Zhang et al., 2005; Hong et al., 2005). Such inactivation of Keap1 allows Nrf2 to accumulate in the nucleus where it forms a heterodimer with other bZIP proteins and transactivates target genes including NQO1, AKR, GST, GCLC and GCLM (Hayes & McMahon, 2001; Motohashi et al., 2002; Kobayashi et al., 2005). Genetic knockdown of Keap1 also increases expression of the ARE-gene battery (Wakabayashi et al., 2003; Devling et al., 2005).

A number of the genes that are regulated by Nrf2 have been linked to drug resistance. For example, the antioxidant GSH, which is primarily regulated by GCL (comprising GCLC and GCLM subunits), has been implicated in resistance of tumour cells to several chemotherapeutic agents, including cisplatin, and the alkylating agent melphalan (Tew, 1994; McLellan & Wolf, 1999; Townsend et al., 2003; Townsend & Tew, 2003; Waxman, 1990). On occasions, high levels of GCLC have been linked to drug resistance (Mulcahy et al., 1995; Ogretmen et al., 1998; Yao et al., 1995). Similarly, over-expression of GST isoenzymes, which catalyse the conjugation of GSH with a wide variety of eletrophilic compounds (Hayes & Pulford, 1995), have been reported in a large number of tumour types (Hayes & Wolf, 1990; Tew, 1994), and these enzymes have been implicated in the development of resistance toward chemotherapeutic agents (Tew, 1994; Townsend et al., 2003). Increases in NQO1 activity have also been shown in certain human lung tumours (Kepa et al., 2003; Schlager et al., 1990; Malkinson et al., 1992; Smitskamp-Wilms et al., 1995). In addition, high levels of manganese superoxide dismutase (MnSOD) (Wong et al., 1995; Kizaki et al., 1993) have been shown to protect cancer cells against the toxic effects of chemotherapeutic agents.

Because drug-metabolising enzymes make a major contribution to determining the sensitivity of tumour cells to anticancer agents, it is important to understand how such genes are regulated and whether modulation of their regulation can lead to improved cancer therapies.

WO2006/128041 teaches the use of RNAi molecules to Nrf2 to reduce expression levels of Nrf2 and sensitise NSCLC cell to anti cancer agents. However, reducing expression of protein using RNAi techniques can suffer from the problem of efficient delivery.

WO 01/57189 teaches the use of antisense RNAi against Nrf2 and dominant-negative mutants, of Nrf2 to augment Fas-induced programmed cell death. Dirumarol and sulfinpyrazone are also shown to antagonise protection conferred by Nrf2 against Fas-induced killing. However, the actual targets of these molecules are not identified.

It is an object of the present invention to provide an assay that allows the identification of agents that may reduce induction of ARE-driven gene expression for use in sensitising cells to other chemical agents.

It is a further object of the invention to provide agents that reduce induction of ARE-driven gene expression as a means of improving therapy of diseases associated with abnormal cell proliferation, such as cancer and psoriasis.

The present invention is based in part on the generation of a sensitive, stable ARE-reporter cell line, comprising multiple concatenated copies of the minimal cis-element found in both rat GSTA2 (Rushmore et al., 1991) and mouse gsta1 (Friling et al., 1992); in the latter gene the element was originally called an EpRE. Previously, Zhu & Fahl (2000) generated a stable ARE-green fluorescent protein (GFP) reporter HepG2 cell line. The reporter construct they employed contained four concatenated copies of the 41-bp ARE-containing promoter sequence from mouse gsta1 ligated to the thymidine kinase promoter driving GFP. However, treatment of the stable HepG2/GFP-B reporter cell line with 90 μM tert butylhydroquinone (tBHQ) resulted in a maximal increase of only 3-fold (Zhu & Fahl, 2000), a level of induction which is not particularly high. Most significantly, the HepG2/GFP-B cell line was used to identify agonists (i.e. chemopreventive inducing agents) rather than to identify antagonists, which inhibit ARE-driven gene expression and may improve therapies. Moreover, the relatively low level of induction observed in the HepG2/GFP-B cell line in response to tBHQ suggests that the cell line would be of little use in identifying antagonists.

### SUMMARY OF THE INVENTION

Methods and products are provided for screening of compounds that can sensitise cells to the effects of toxic and antiproliferative drugs. Such compounds may themselves affect cell death/induction of apoptosis, or result in rendering effective treatment with other agents that would otherwise be ineffective due to, for example, detoxification of the agent, sequestration of the agent, removal of the agent from the cell, or simply intrinsic resistance to action of the agent. The methods comprise adding the compound in an appropriate medium to ARE responsive cells into which has been stably introduced a genetic construct comprising an ARE response element with a reporter gene under the transcriptional regulation of the ARE response element and a promoter.

In a first aspect there is provided an agent which is capable of down-regulating Nrf2 activity for the manufacture of a medicament for use in therapy.

The agent preferably down-regulates transactivation of gene expression by Nrf2 and in particular transactivation of genes which comprise an antioxidant response element(ARE) in their promoter.

The agent may find application in treating diseases associated with abnormal cell proliferation, such as cancer and psoriasis.

The present inventors have identified that by down-regulating the transactivation activity of Nrf2, cells can become sensitised which can lead to cell death. For example, the effects of some cytotoxic agents can be reduced by the ability of Nrf2 to transactivate genes having an ARE. By down-regulating Nrf2 activity, the efficacy of such cytotoxic drugs can increase, with the possible advantages of shorter periods of treatment and/or less cytotoxic drug being required.

Unlike some prior art teaching, the present invention is concerned with small molecule chemical antagonists of Nrf2 activity. The antagonists do not generally have an effect on Nrf2 expression or mRNA levels, but rather on the activity of Nrf2 itself. This is quite different to genetic techniques designed at reducing Nrf2 expression, such as by the use of RNAi or antisense technology. The present invention is therefore concerned with the use of nucleic acid based inhibitors of Nrf2.

The agents of the present invention will typically have a molecular weight of less than about 1000-2000 Mn, such as less than 750 mW.

The present inventors have carried out screens of small molecules and observed that retinoic acid and certain derivatives thereof, as well as other chemical agents, are potent agents which are capable of decreasing induction of ARE-driven gene expression.

Thus, in a further aspect there is provided use of a retinoid for the manufacture of a medicament for use in treating diseases associated with abnormal cell proliferation wherein the retinoid sensitises an abnormally proliferating cell in a host by way of down-regulating ARE-driven gene expression.

Typically, the retinoid down-regulates the transactivation of gene expression by Nrf2.

By retinoid is meant retinoic acid, in the various stereoisomeric forms, including all trans-retinoic acid, 9-cis retinoic acid and 13-cis retinoic acid as well as acitration retinal and retinol and salts such as an acetate. A general structure identifying a number of potential retinoids which can be suitable in the present invention is shown below:

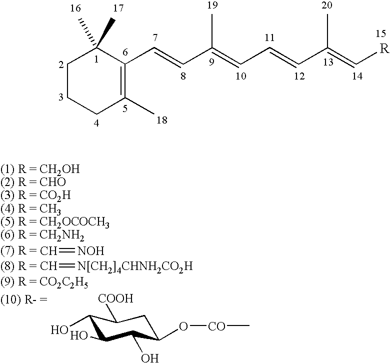

In a screen of a commercially available chemical library (Maybridge Chemical Corp.) a further compound was identified as having significant activity in down-regulating ARE-driven gene expression. Thus, the present invention also extends to the use of compounds according to formula (I) for the manufacture of a medicament for use in treating diseases associated with abnormal cell proliferation wherein the compound of formula (I) sensitises an abnormally proliferating cell in a host by way of down-regulating ARE-driven gene expression.

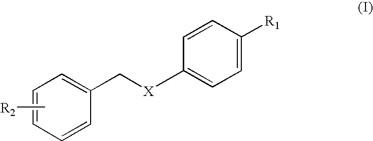

wherein X is C, O, N or S; R₁, is C₁-C₄ alkyl, C₁-C₄(OH), COOH, C(═CH₂)CH₃, C(═O)CH₃, CH(CH₃)₂, C(CH₃)₃; and R₂ is independently selected from, at each available position, H, halo, C₁-C₄ alkyl, OH or NH₂.

Preferably X is O. Preferably R₁ is C(═O)CH₃. Preferably R₂ is halo and h, more preferably halo at positions 3 and 4, especially chlorine.

A particularly preferred compound is where X is O, R₁ is C(═))CH₃ and R₂ is H at positions 2, 5 and 6 and Cl at positions 3 and 4.

In a further aspect there is provided a pharmaceutical composition comprising, or consisting essentially of, as active ingredients, an agent capable of down-regulating Nrf2 activity, a retinoid and a chemotherapeutic agent.

It is understood that the retinoid serves to down-regulate ARE-driven expression, thereby sensitising the cell to apoptosis or treatment by another agent, such as a alkylating agent or a redox cycling compound and thereby improving efficacy of the chemotherapeutic agent when treating cancer, for example. Thus, the use of a retinoid in combination with another agent enables the treatment to be more effective and/or allows for less of the other agent to be administered to a subject.

Suitable chemotherapeutic agents for treating cancer include the alkylating agents cisplatin, melphalan, chlorambucil, mitrozantrone and BCNU; and redox-cycling agents such as etopside. Other agents that may be of use in combination with a sensitising agent have been hereinbefore described.

The pharmaceutical composition may further comprise a redox controlling agent, such as BSO, in order to control the redox status of the cell, as this may also improve the efficacy of the chemotherapeutic agent.

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersable granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectible preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

In a further aspect there is provided a method a method of treating a patient suffering from a disease associated with abnormal cell proliferation, comprising the step of administering to the subject an effective amount of an agent which is capable of down-regulating transactivation of gene expression by Nrf2.

In a further aspect, there is provided a method of treating a patient suffering from a disease associated with abnormal cell proliferation, comprising the step of administering to the subject an amount of a retinoid or compound according to formula I, capable of reducing ARE-driven gene expression and an agent, wherein in combination the retinoid or compound according to formula I and agent serve to ameliorate the cell proliferation, such as by inducing cell death.

It is understood that the retinoid or compound according to formula I and other agent may be administered concurrently or separately. If added separately, the retinoid or compound according to formula I will generally be administered before the other agent.

In a further aspect there is provided a method of screening for agents which down-regulate induction of ARE-driven gene expression, for use in sensitising cells, comprising the steps of:

a) providing in vitro a cell which is capable of driving an antioxidant response, wherein the cell comprises an ARE-reporter gene construct, comprising a reporter gene located downstream of multiple concatenated ARE sequences;

b) contacting a test agent to be screened with said cell; and

c) detecting whether or not said agent is capable of decreasing induction or decreasing expression of the reporter gene, in comparison to a cell to which the test agent has not been added.

The present screen finds application in identifying agents which are able to sensitise cells and which may be of use in treating diseases associated with abnormal cell proliferation, such as cancer and psoriasis.

Sensitisation of the cells may itself have a therapeutic effect, as the cells may have increased spontaneous sensitivity to apoptosis resulting from alterations in redox balance, but often sensitisation will lead to the ability or improve the ability of other agents to treat the undesirably proliferating cells. Such agents may include chemotherapeutic agents conventionally used to treat, for example, cancers where it is envisaged that the sensitisation will improve or enhance efficiency of their action.

However, in view of the effect of such sensitising agents on the Nrf2 pathway, other agents may be of utility in treating diseases associated with abnormal cell proliferation once the proliferating cells have been sensitised. For example, agents that may become therapeutically effective following antagonism of Nrf2 include those compounds that are detoxified by enzymes normally regulated by Nrf2; it is envisaged that compounds that induce oxidative stress, compounds which are transported into/out of a cell via MRP2, or related efflux pump, such as cisplatin, chlorambucil, cyclophosphamide, doxorubicin, methotrexate and vincristine (Wawabe et al., 1999; Smitherman et al., 2004; Vlaming et al., 2006) will fall into this category. The invention will also allow novel antitumour agents to be developed that would normally be detoxified via Nrf2-dependent genes. In this case the novel agent will be applied with an Nrf2 antagonist or a bifunctional molecule could be synthesized that possesses both anticancer properties plus Nrf2 inhibitory activity.

Conveniently, the ARE sequence used is from that in the rat GSTA2 (5′-GTG ACA AAG CA-3′) and/or mouse gsta1 genes.

Desirably the cell is a tumour cell, although any mammalian cell may be appropriate, which is capable of driving an antioxidant response. Suitable cells include MCF7, HepG2, CHO and Hepa1 and HaCaT, with MCF7 being preferred for reasons of sensitivity.

Contacting of the test agent with the cell may be carried out by any suitable means, such as adding the test agent to the culture medium in which the cell is growing.

Induction of the reporter gene may be enhanced by addition of an activating agent such as tBHQ, sulforaphane, diethyl maleate or β-naphthoflavone, in order to more easily identify agents which are able to down-regulate or decrease expression of the ARE-driven reporter gene, or where the contributive activity in the cell line is inherently low. The activating agent will generally be added to the cells before the test agent.

It may also be possible to activate Nrf2 by down regulating expression of Keap1 using antisense or RNAi; techniques. Activated Nrf2 will then act on the ARE sequence causing induction of reporter gene expression. It may similarly be possible to increase the activity of Nrf2 by down-regulating the expression of negatively-acting competing transcription factors such as Bach1, Bach2, cFos and small Maf.

Detection of an effect the test agent has on the induction of the ARE-driven reporter gene will depend on the reporter gene being employed, but suitable techniques are well known to the skilled addressee. Typical reporter genes include GFP and related fluorescent proteins, luciferase, β-galactosidase, chloramphenicol acetyl transferase and the like. Any assay that detects a product of the reporter gene, either by directly detecting the protein encoded by the reporter gene or by detecting an enzymatic product of a reporter gene-encoded enzyme, is suitable for use in the present invention. Assays include colorimetric, fluorimetric, or luminescent assays or even, in the case of protein tags, radioimmunoassay or other immunological assays. Many of these assays are commercially available.

Typically a comparison or control experiment is used to ascertain a level or degree of reporter activity, in the absence of the test agent, so that the effect of the test agent can easily be detected. By measuring the effect of the candidate compound on the level of signal observed, as compared to a basal level, one can evaluate the potential of the compound as a sensitising agent for use in the treatment of cancer.

Conveniently, the method is carried out in a multiwell format, e.g. 24, 48, 96 well plates may be used in order to allow many such tests methods to be carried out simultaneously for multiple compounds and optionally using automated or semi-automated means.

In a further aspect there is provided a cell for use in screening agents for an effect on ARE-driven gene expression, wherein the cell is a human mammary MCF7 cell containing an ARE reporter construct that comprises a reporter gene downstream of multiple concatenated copies of the ARE sequence from the rat GSTA2 and mouse gsta1 genes.

Preferably, the reporter gene is a luciferase gene, such as the firefly or Renilla luciferase gene. The reporter gene may be under further control of a minimal promoter immediately upstream of the reporter gene, but downstream of the ARE sequences. Typical minimal promoters include the SV40 promoter and thymidine kinase promoter and the ARE sequence may be immediately adjacent to the promoter sequence or spaced therefrom by up to 10 kb.

The multiple concatenated ARE sequences are located head-to-tail, in series, upstream of the reporter gene. Conveniently the number of copies is 4, 5, 6, 7 or 8, or even more, each of which is separated by a short linker sequence, such as 5′-CCC-3′ (the size of the linker is not important). Preferably the numbers of copies are 6-8 or more. Preferred sequences are shown in Table 1, particularly with respect to 6 and 8 copies.

The construct may be prepared in accordance with conventional ways, introducing each of the components of the construct into a plasmid by employing convenient restriction sites, PCR (polymerase chain reaction) to introduce specific sequences at the termini, which may include providing for restriction sites, and the like.

After the reporter construct has been prepared, it may be introduced into the cells by any suitable means. Methods for introducing the ARE-driven reporter construct into the cells or cell lines include transfection, complexing with cationic compounds, lipofection, electroporation, and the like. The cells may be expanded and then screened for the continual presence of the reporter construct. Where an antibiotic resistance gene has been introduced along with the reporter construct, the cells may be selected for antibiotic resistance and the antibiotic resistant cells then screened for luminescence under appropriate conditions. In the absence of the antibiotic resistance, the cells may be directly screened for luminescence. Conveniently, the assay for luminescence is performed on a lysate using conventional reagents.

If the reporter gene is luciferase, the luminescence may be determined in accordance with conventional commercial kits. The cells may be distributed in multiwell plates that can be accommodated by a luminometer. A known number of cells may be introduced into each one of the wells in an appropriate medium, the candidate compound added, and the culture maintained for at least 12 hours, more usually at least about 24, and not more than about 60 hours, particularly about 48 hours. In conjunction with the candidate compound, an inducing compound, e.g. tBHQ, sulforaphane, diethyl maleate or β-naphthoflavone may also be added. The culture is then lysed in an appropriate buffer, using a non-ionic detergent, e.g. 1% triton X-100. The cells are then promptly assayed. The concentration of the inducing agents will vary depending upon the nature of the agent, but will be sufficient to induce expression. The concentration of tBHQ, for example, will generally be in the range of about 1-100 μM, preferably about 50 μM.

Any other technique for detecting the level of luminescence may be used. The particular manner of measuring luminescence is not critical to the invention.

The types of test agents include small chemical entities and peptide molecules.

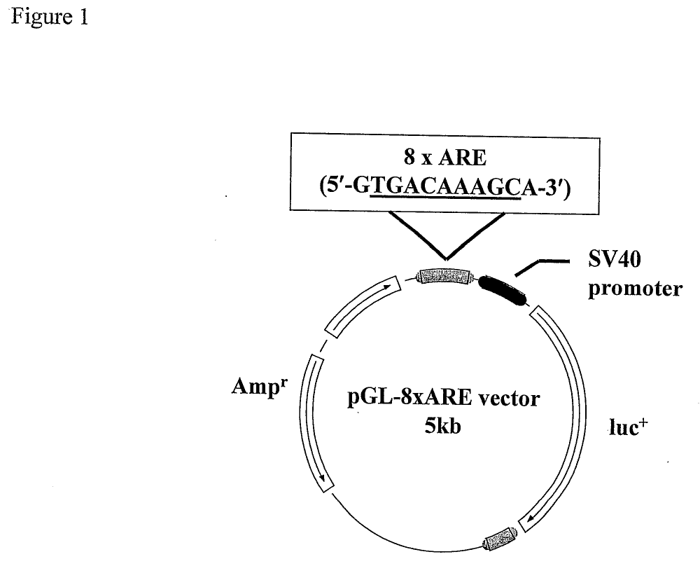

The present invention will now be further described with reference to FIGS. 1-13 presented below that show data relevant to the invention.

FIG. 1: Map of the ARE-driven reporter plasmid.

The cartoon shows the pGL-8xARE vector. A single ARE from the rat GSTA2 and mouse gsta1 gene promoters is presented above the plasmid with the ‘core’ sequence shown underlined. Note, the reporter plasmid contains 8 tandemly arrayed copies of the 5′-GTGACAAAGCA-3′ sequence, each connected with a 5′-CCC-3′ linker (as shown in Table 1). The size of the linker can be varied.

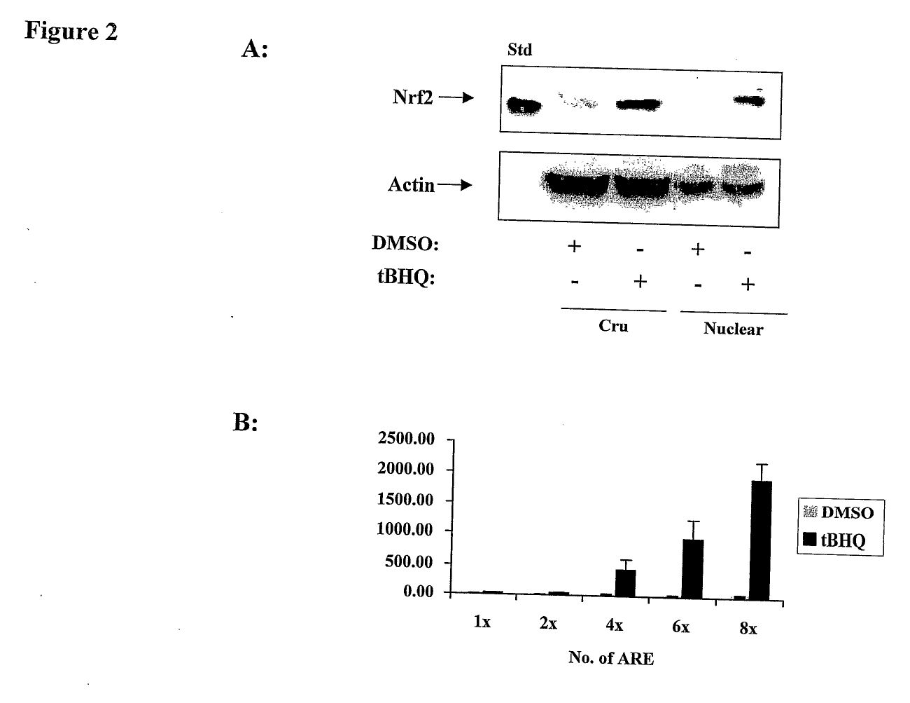

FIG. 2: Correlation between ARE copy number and induction of reporter gene activity by tBHQ in MCF7 cells.

(A) MCF7 cells were cultured in DMEM supplemented with antibiotics containing DMSO or 10 μM tBHQ for 24 h. Thereafter the cells were harvested. Portions, 60 μg of protein, of whole-cell extracts (Cru) and portions, 20 μg of protein, of nuclear extracts (Nuclear) were subjected to 7% SDS-PAGE and the expression of Nrf2 protein was measured by western blotting. Std, 1 ng recombinant his-mNrf2. The blots shown represent the results from at least three separate experiments.

(B) MCF7 cells were seeded at 2×10⁵ cells/well in 24-well plates, transfected with the pGL3-nxARE constructs, treated with 50 μM tBHQ. Luciferase reporter activity was determined 18 h later. The data represent the results of three separate experiments. Each treatment in each experiment has at least three replicates.

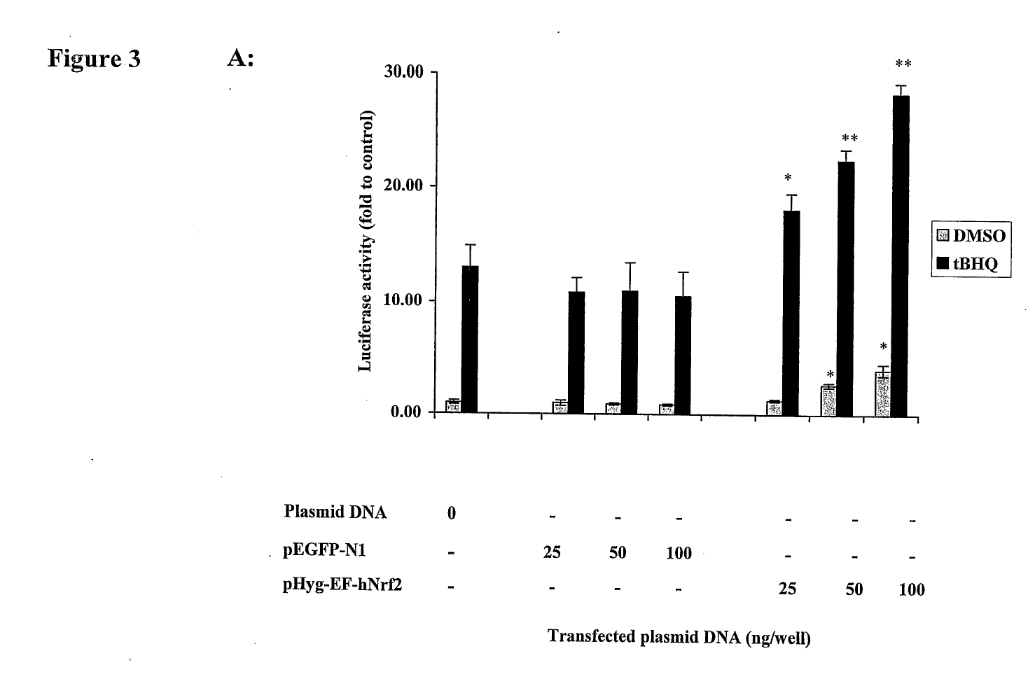

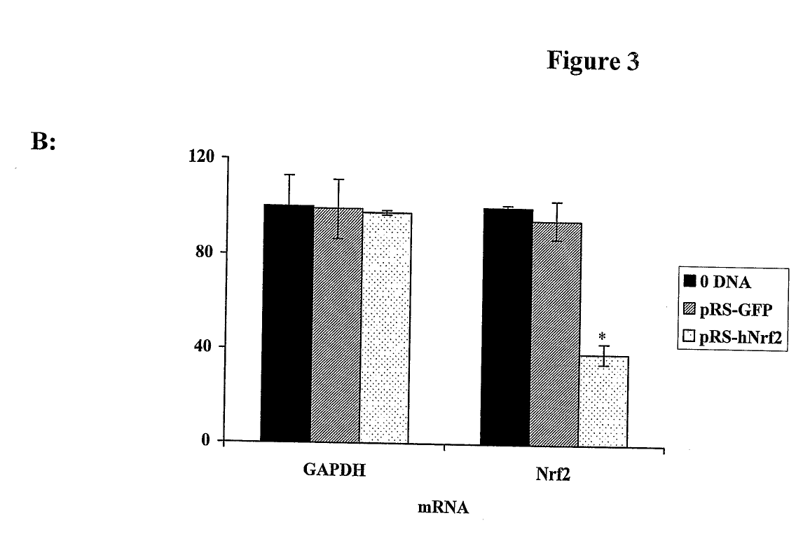

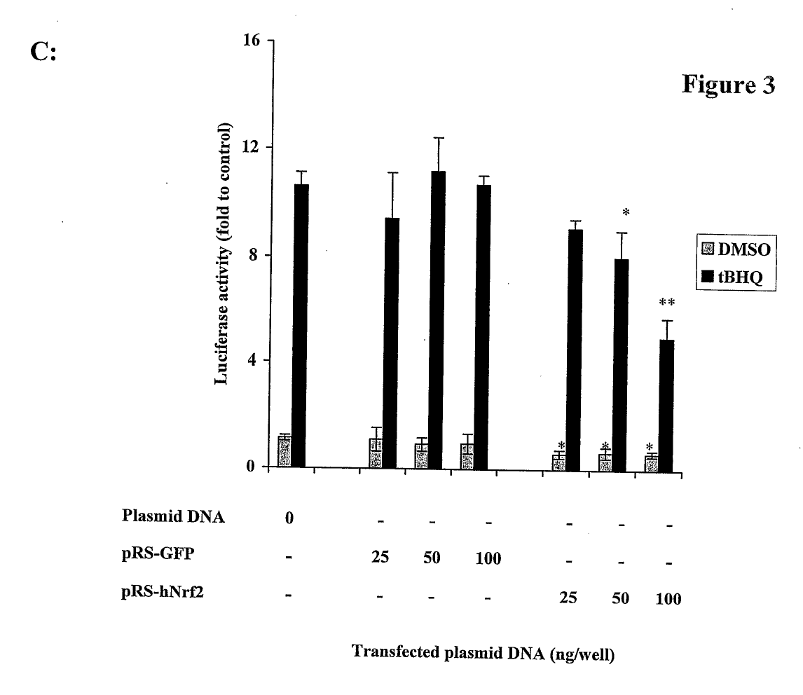

FIG. 3: Luciferase reporter activity in AREc32 cells is mediated by Nrf2.

(A) Over-expression of Nrf2 in AREc32 cells increased both the basal and the inducible luciferase reporter activity. AREc32 cells were seeded in a 96-well plate at 1.5×10⁴ cells/well, and transfected with either 25, 50 or 100 ng/well of pHyg-EF-hNrf2. The same amount of pEGFP-N1 was transfected as negative control. After transfection (24 h), the cells were treated with either DMSO alone or 10 μM tBHQ (in DMSO). The luciferase activity was assayed. Control, DNA was absent and the transfection reagent was only added to the cells and treated with DMSO for 24 h.

(B) Knockdown of Nrf2 by RNAi vector in the AREc32 cell line. The AREc32 cells were seeded in 100 mm dishes at 8×10⁶ cells/dish in the growth medium. Twenty-four h later, the cells were transfected with 24 μg pRS-hNrf2 or pRS-GFP per plate. After a further 24 h had elapsed, total RNA was extracted from the cells and levels of Nrf2 and GAPDH mRNAs were measured by TaqMan RT-PCR. The level of 18S rRNA was used as an internal standard. The mRNA level from the cells mock transfected (control) was set at 100%.

(C) Suppression of Nrf2 expression in AREc32 cells reduces the basal and inducible luciferase reporter activity. In a parallel experiment to that shown in panel (B), AREc32 cells were seeded in a 96-well plate at 1.5×10⁴ cells/well, transfected with 25, 50 and 100 ng/well pRS-hNrf2. The same amount of pRS-GFP was transfected as negative control. Twenty-four hours after the transfection, the cells were treated with DMSO or 10 μM tBHQ. Luciferase activity was assayed. Control, DNA was absent and the transfection reagent was only added to the cells and treated with DMSO for 24 h. The histograph shows luciferase activity as mean±S.D. from triplicate samples. Each treatment in each experiment has at least three replicates. The significance of the differences between luciferase activity from cultures transfected with pRS-hNrf2 or pEGF-Nrf2 and the control was assessed by unpaired student's t-test. (*)p<0.05; (**)p<0.005.

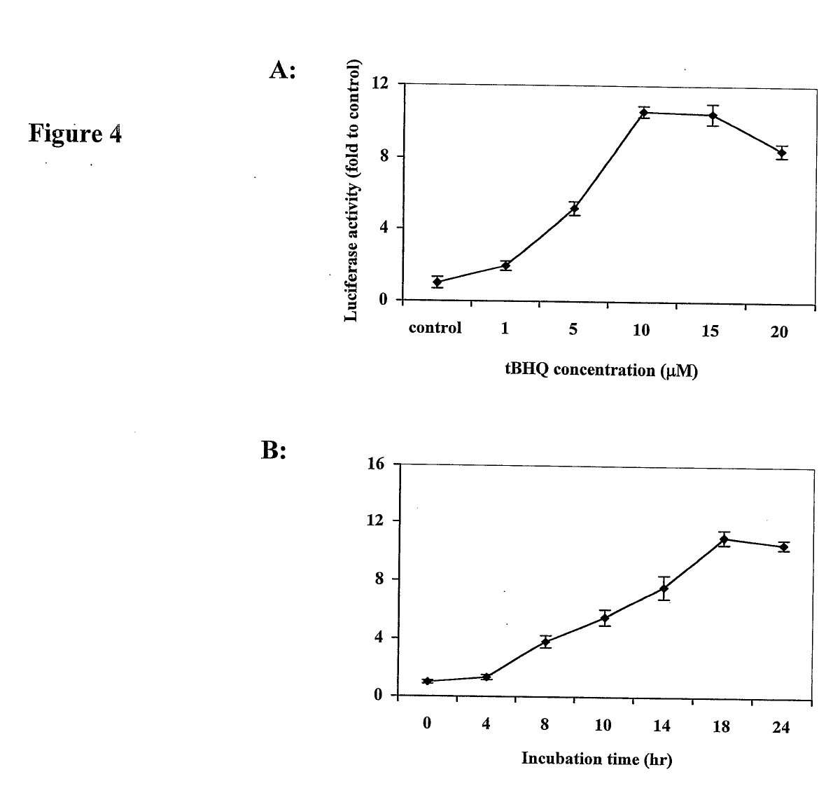

FIG. 4: Induction of ARE-driven reporter gene activity by tBHQ in MCF7 cells in a time- and dose dependent manner.

Cells were seeded in a 96-well plate at 1.2×10⁴ cells/well in the growth medium. After 24 h recovery, the culture medium was replaced with fresh DMEM supplemented with antibiotics containing 1-20 μM tBHQ. The cells were then incubated for between 4-24 h, and assayed for luciferase activity. The value of luciferase activity of cells treated with DMSO (0.1% v/v) was set at 1.

Panel (A) shows the dose response of luciferase induction following treatment of AREc32 cells for 24 h with various concentrations of tBHQ.

Panel (B) shows the time course of luciferase induction following treatment of AREc32 cells with 10 μM tBHQ.

The data shown represent the results of three separate experiments. Each treatment in each experiment has at least three replicates.

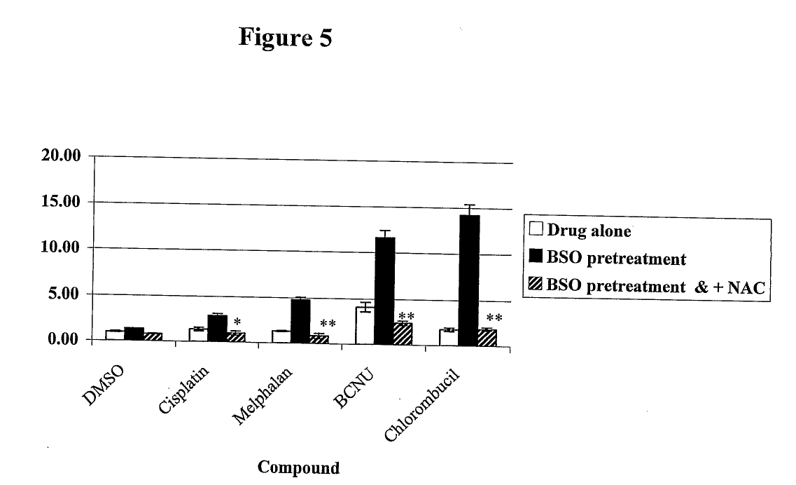

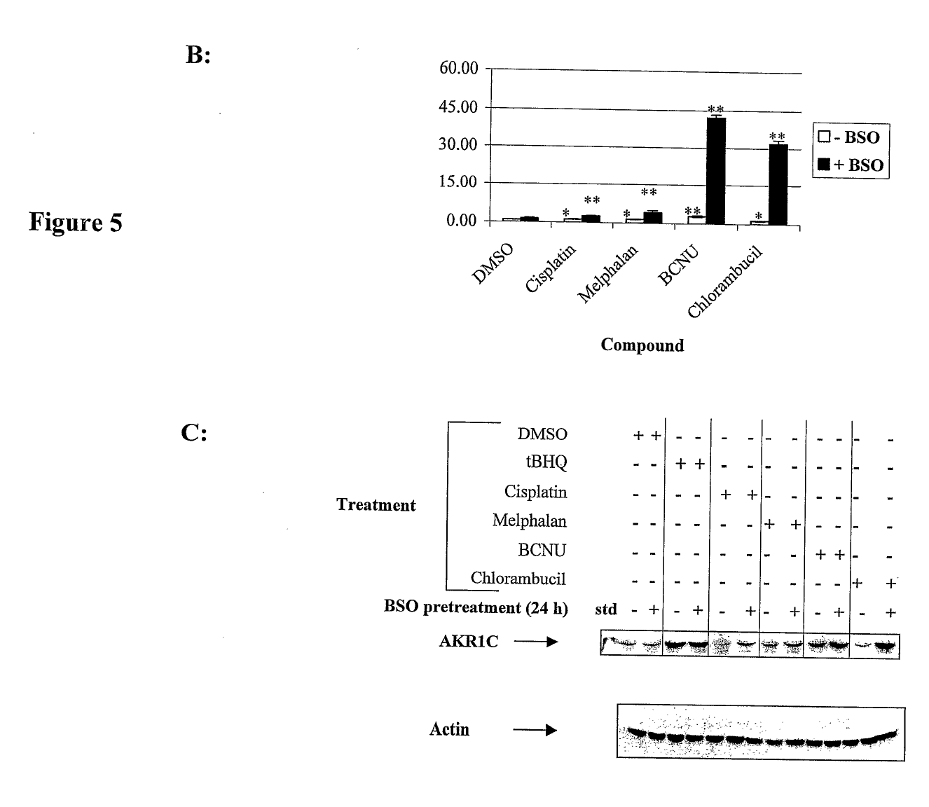

FIG. 5: Induction of reporter gene activity and AKR1C in AREc32 cells by anticancer drugs is redox dependent.

(A) BSO enhanced the induction of luciferase activity in AREc32 cells by anticancer drugs. AREc32 cells were seeded in a 96-well plate at 0.4×10⁴ cells/well. After 24 h recovery, the culture medium was replaced with growth medium containing 50 μM BSO; an equal volume of PBS was added to the cells that were not pre-treated with BSO. After a further 24 h, during which time the BSO could deplete GSH, the culture medium was replaced with fresh DMEM supplemented with antibiotics containing either DMSO (control), or 10 μM cisplatin, or 20 μM melphalan, or 100 μM BCNU, or 100 μM chlorambucil, all with or without 5 mM NAC, and incubated for 24 h. The cells were assayed for luciferase activity. The value of control cells treated with DMSO was set at 1. The reporter gene activity data shows mean±S.D. from triplicate samples. The significance of the differences between luciferase activity from cultures exposed to the anticancer agents with NAC and cultures treated with the anticancer agents alone was assessed by unpaired student's t-test. This represents the results of three separate experiments. (*)p<0.05; (**)p<0.005.

(B) AKR1C mRNA was induced by anticancer drugs in a redox-dependent manner. AREc32 cells were seeded in 100 mm dishes at 2×10⁶ cells/dish in the growth medium. After 24 h recovery, the culture medium was replaced with growth medium containing 50 μM BSO. Twenty-four h later, the culture medium was replaced with fresh DMEM supplemented with antibiotics containing either DMSO, 10 μM tBHQ, 20 μM melphalan, 10 μM cisplatin, 100 μM BCNU, or 100 μM chlorambucil and incubated for a further 24 h before the cells were harvested. The expression of AKR1C mRNA was measured by TaqMan analysis. The mRNA level of AKR1C of cells treated with DMSO (control) was set at 1. The significance of the differences between AKR1C mRNA level from cultures exposed to the anticancer agents and those exposed to DMSO was assessed by unpaired student's t-test. The data represent means of two separate experiments, and each treatment in each experiment has three replicates. (*)p<0.05; (**)p<0.005

(C) In a parallel experiment to that shown in panel (B), 30 μg of protein from whole-cell lysates were resolved using SDS-PAGE. The expression of AKR1C was measured by western blotting with antibody specific to AKR1C. The blots shown represent the results from three separate experiments.

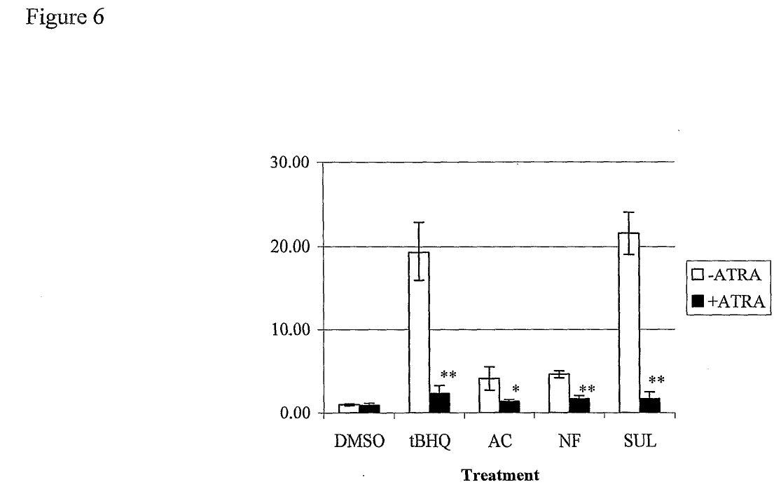

FIG. 6: All trans-retinoic acid suppresses the induction of ARE-driven luciferase activity.

AREc32 cells were seeded in a 96-well plate at 1.2×10⁴ cells/well in the growth medium. After 24 h recovery, the culture medium was replaced with fresh DMEM supplemented with antibiotics containing 10 μM tBHQ, 10 μM SUL, 10 μM acrolein or 10 μM β-naphthoflavone (NF), and 1 μM all trans-retinoic acid (ATRA) was added to the medium concomitantly with the inducing agents. The cells were incubated with the various inducing agents, with and without ATRA, for 24 h before they were harvested and luciferase activity measured. The value of luciferase activity of cells treated with DMSO (0.1% v/v) was arbitrarily set at 1, and the data presented shows mean±S.D. from triplicate samples. The significance of the differences between luciferase activity from cultures exposed to the inducers with and without the presence of ATRA was assessed by unpaired student's t-test. This represents the results of three separate experiments. (*)p<0.05, (**)p<0.005;

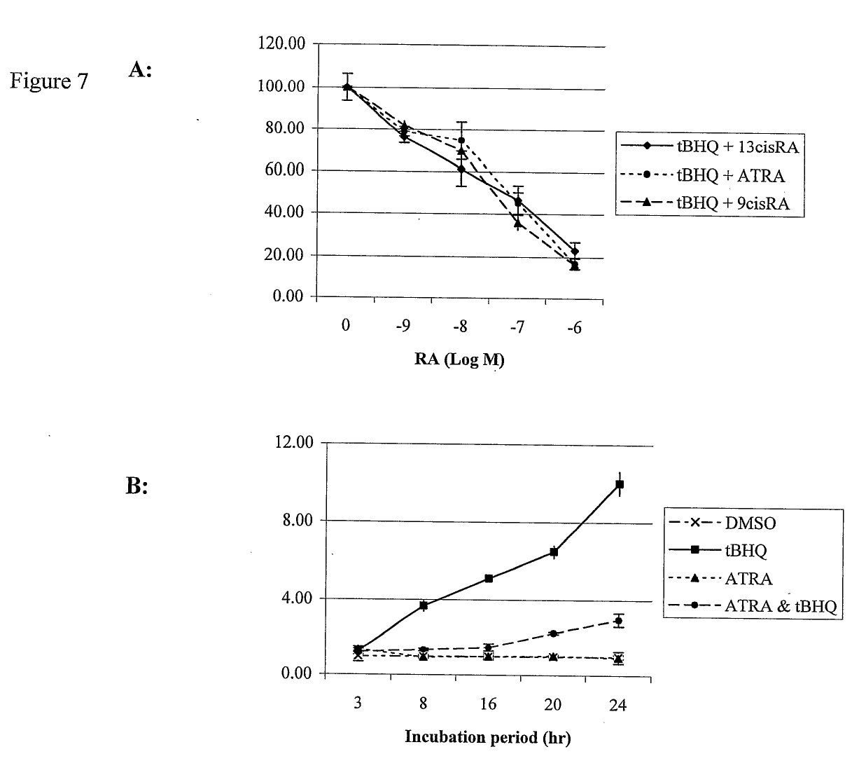

FIG. 7: Concentration- and time-dependent inhibition by ATRA on the induction of ARE reporter activity by tBHQ in AREc32 cells

(A) To determine the dose response of inhibition by retinoic acids of inducible ARE-driven gene expression, AREc32 cells were seeded in a 96-well plate at 1.2×10⁴ cells/well in the growth medium. After 24 h recovery, the culture medium was replaced with fresh DMEM supplemented with antibiotics containing 10 μM tBHQ along with various concentrations (10⁻⁹ M to 10⁻⁶ M) of either ATRA, 9-cisRA or 13-cisRA. Thereafter the cells were incubated for a further 24 h before being harvested and luciferase activity measured. The value of luciferase activity of cells treated with 10 μM tBHQ alone, without retinoic acid (control), was set at 100%.

(B) To establish the time course of inhibition by all trans-retinoic acid (ATRA) of inducible ARE-driven gene expression, AREc32 cells were seeded in a 96-well plate at 1.2×10⁴ cells/well in the growth medium. After 24 h recovery, the culture medium was replaced with fresh DMEM supplemented with antibiotics containing 10 μM tBHQ, or 1 μM ATRA, or 10 μM tBHQ plus 1 μM ATRA, and further incubated for 4-24 h. The value of luciferase activity of cells treated with DMSO (0.1% v/v) (control) at each time point was arbitrarily set at 1. The data shown represent the results of three separate experiments. Each treatment in each experiment has at least three replicates.

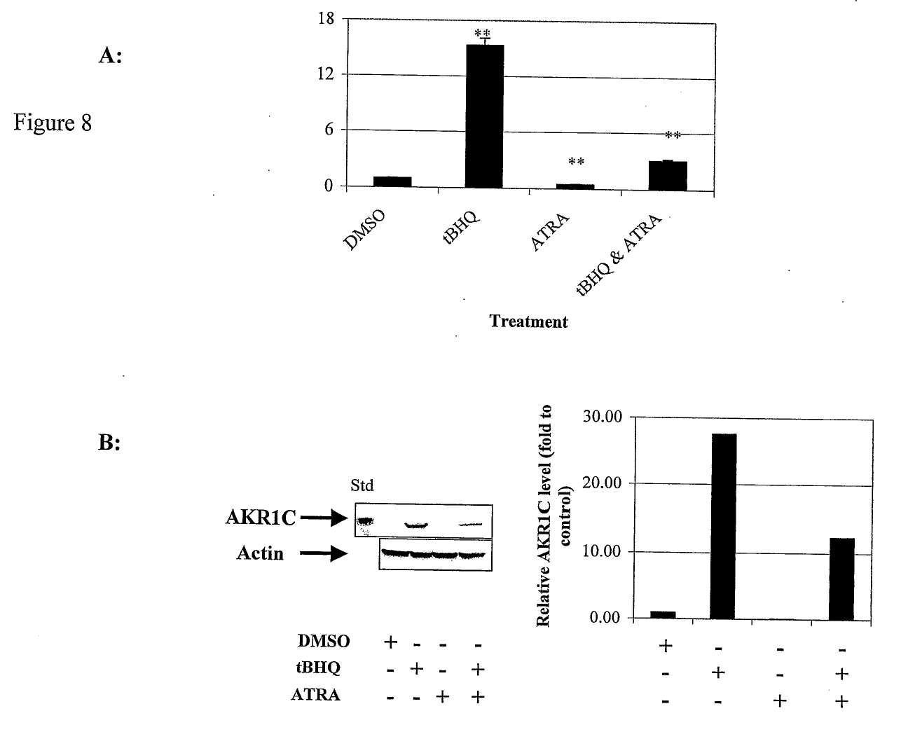

FIG. 8: Induction of endogenous AKR1C by tBHQ was inhibited by ATRA in AREc32 cells.

AREc32 cells were seeded in 100 mm dishes at 2×10⁶ cells/dish in the growth medium. After 24 h recovery, the culture medium was replaced with fresh DMEM supplemented with antibiotics containing either DMSO, 10 μM tBHQ, 1 μM ATRA, or 10 μM tBHQ plus 1 μM ATRA and incubated for a further 24 h.

(A) After 24 h treatment, total RNAs were extracted. The mRNA level of AKR1C was measured by TaqMan analysis. The level of 18S rRNA was used as an internal standard. Control, cells were treated with DMSO only. The TaqMan data shows mean±S.D. from triplicate samples and represents the results of three separate experiments. The significance of the differences between mRNA levels from cultures with the different treatment and the control was assessed by unpaired student's t-test. (*)p<0.05.

(B) Whole-cell extracts were prepared from the cells treated with different agents. The expression of AKR1C and actin were measured by western blotting. The blots shown represent the results from three separate experiments.

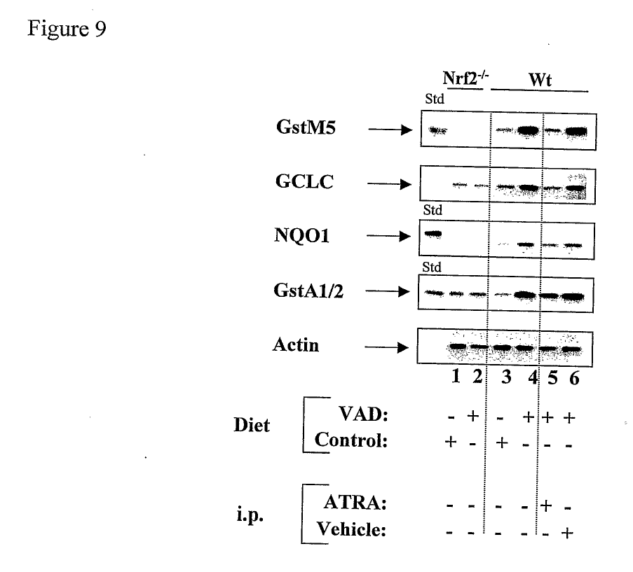

FIG. 9: All trans-retinoic acid suppressed the expression of GST, GCLC and NQO1 in the small intestine of Nrf2 (+/+) mice.

Wild-type (nrf2^{+/+}) and knockout (KO, nrf2^{-/-}) mice, 8 weeks old, were placed on control or vitamin A deficient (VAD) diet for six weeks as described in “Materials and Methods II”. Portions (5 μg protein) of crude extracts from small intestine of wild-type and KO mice were subjected to Western blotting with specific antibodies against NQO1, GstM5, GstA1/2 and GCLC. Each lane contains a sample from an individual mouse. In one series of experiments, all trans-retinoic acid was administered (i.p. at 10 mg/Kg body weight) to wild-type animals on VAD diet for the last 2 weeks of the experiment. These animals were sacrificed and immunoblotting for GST, GCLC and NQO1 performed as before.

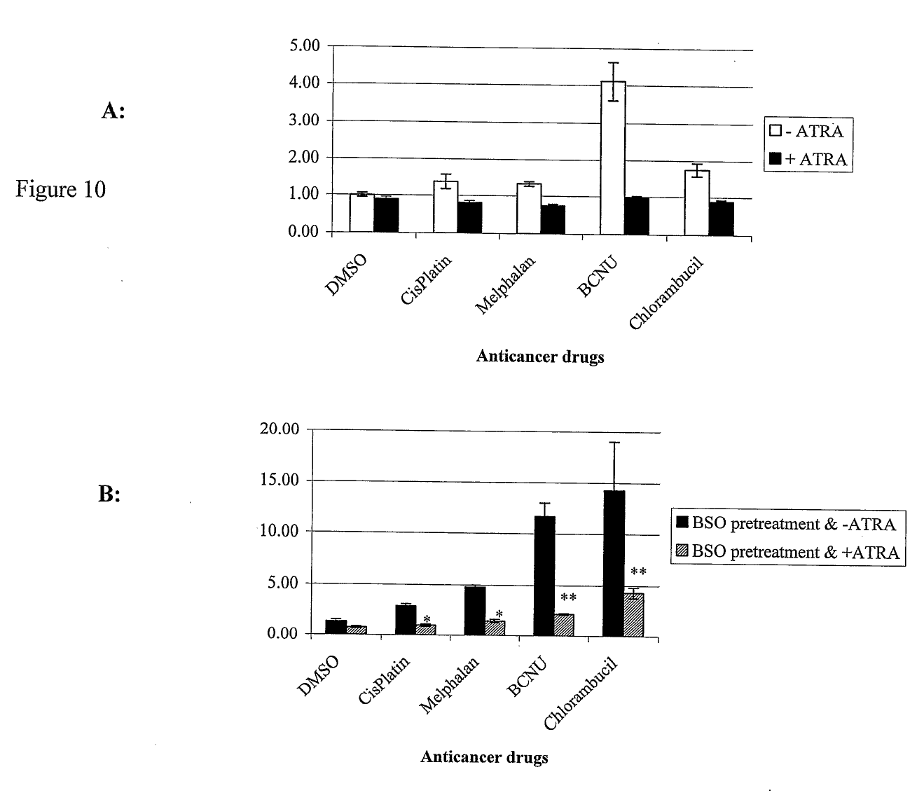

FIG. 10: ATRA repressed the induction of luciferase reporter activity by anticancer drugs in AREc32 cells

(A) AREc32 cells were seeded in a 96-well plate at 1.2×10⁴ cells/well. After 24 h recovery, the culture medium was replaced with fresh DMEM supplemented with antibiotics containing either DMSO (control), 10 μM cisplatin, 20 μM melphalan, 100 μM BCNU, or 100 μM chlorambucil with or without 1 μM ATRA, incubated for 24 h. The cells were assayed for luciferase activity as detailed in the Materials and Methods. The luciferase value obtained from DMSO treated AREc32 cells was set at 1.

(B) AREc32 cells were seeded in a 96-well plate at 0.4×10⁴ cells/well. After 24 h recovery, the culture medium was replaced with growth medium containing 50 μM BSO; an equal volume of PBS was added to the cells without BSO pre-treatment. Following 24 h incubation with 50 μM BSO, to allow depletion of intracellular GSH, the medium was replaced with fresh DMEM supplemented with antibiotics containing either DMSO (control), 10 μM cisplatin, 20 μM melphalan, 100 μM carmusitine, or 100 μM chlorambucil with or without 1 μM ATRA, incubated for 24 h. The cells were assayed for luciferase activity as detailed in the Materials and Methods. The value of control cells treated with DMSO was set at 1.

The data shows mean±S.D. from triplicate samples. The significance of the differences between luciferase activity from cultures exposed to the anticancer agents with ATRA and cultures treated with the anticancer agents alone was assessed by unpaired student's t-test. This represents the results of three separate experiments. (*)p<0.05; (**)p<0.005).

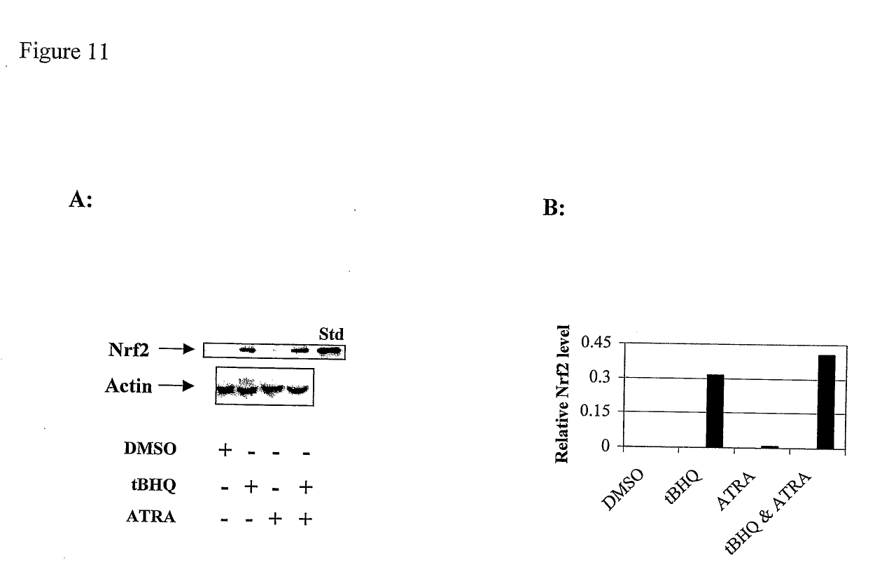

FIG. 11: ATRA did not block nuclear translocation of Nrf2.

Nuclear extracts were prepared from AREc32 cells that had been treated for 24 h with either 10 μM tBHQ, 1 μM ATRA, or 10 μM tBHQ plus 1 μM ATRA. Portions (20 μg of protein) from nuclear extracts were loaded on 7% SDS-PAGE, blotted onto nitrocellulose transfer membrane, and the presence of Nrf2 probed using an antibody against the mouse protein. In panel A, the blot shown represents a typical result of at least three separate experiments. In panel B, the blot shown in A has been densitometrically scanned.

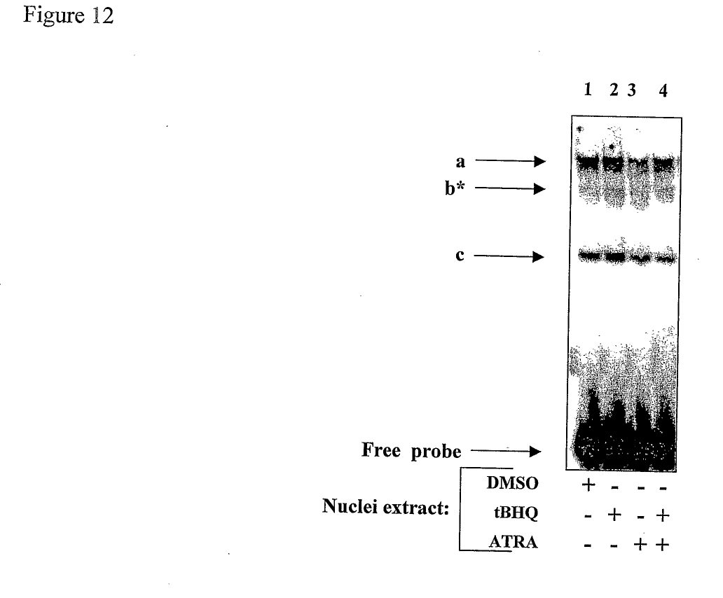

FIG. 12: ATRA reduced the binding of protein complexes to an ARE sequence.

Nuclear extracts (10 μg of protein) from AREc32 cells that had been incubated for 24 h with 10 μM tBHQ, in the absence or presence of 1 μM ATRA, were analysed for their ability to bind an ARE by EMSA. A 200-fold excess of unlabeled ARE was used to monitor the specificity of binding. Arrows indicate the specific bands of DNA-protein complexes. Results represent three separate experiments.

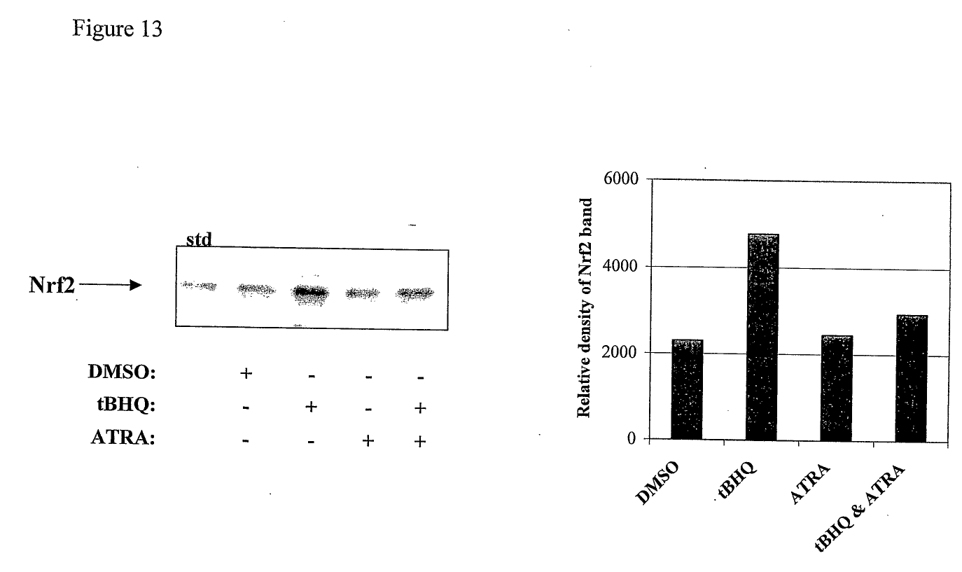

FIG. 13: ATRA interfered with the binding of Nrf2 to an ARE sequence.

Nuclear extracts were prepared from AREc32 cells that had been incubated for 24 h with 10 μM tBHQ in the absence or presence of 1 μM ATRA. Portions (100 μg protein) of the nuclear extracts were incubated with a biotinylated ARE oligonucleotide, and a pull-down assay was performed as detailed in Materials and Methods. The pull-down beads were subjected to SDS-PAGE and immunoblotted with specific anti-Nrf2 antibody. Mock oligonucleotides were included as a negative control.

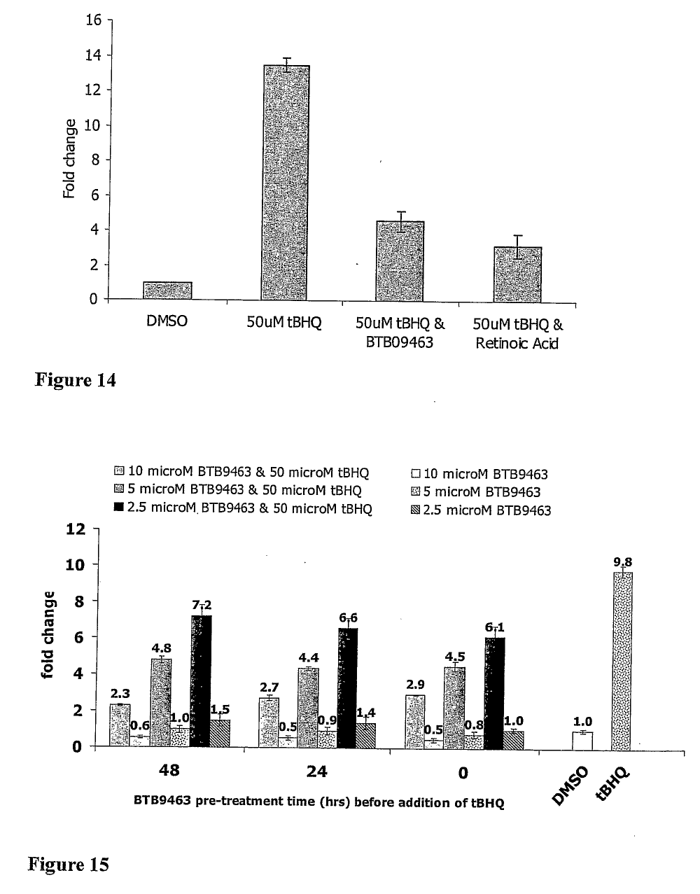

FIG. 14 shows BTB09463 and Retinoic acid can antagonise the tBHQ induced expression of luciferase in the ARE-reporter cell line AREc32.

FIG. 15 shows pre-treatment of AREc32 cells with BTB09463 for up to 48 hrs before the addition of tBHQ has the same inhibitory effect on luciferase expression as concomitant dosing.

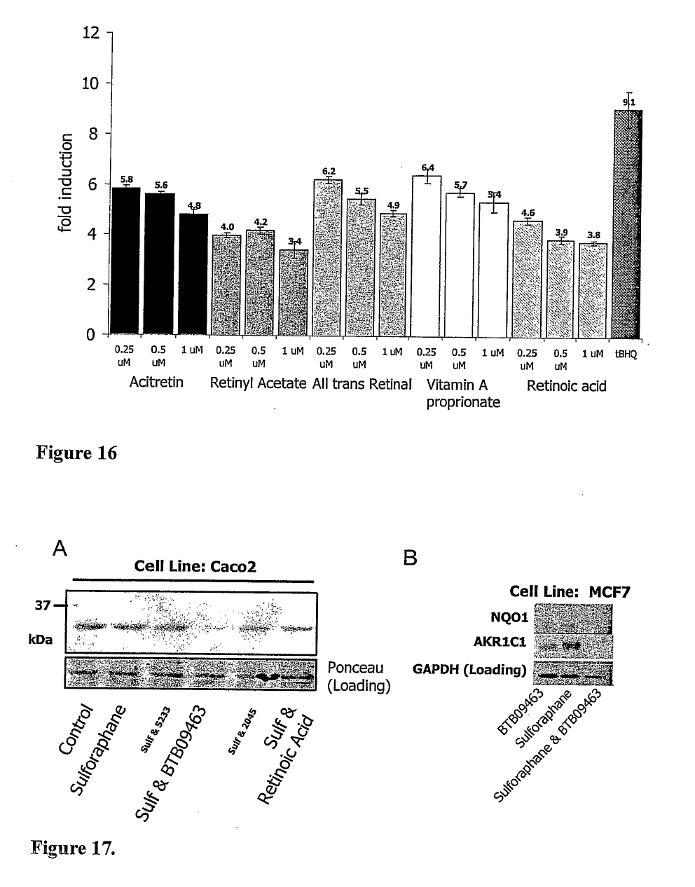

FIG. 16 shows that Several retinoids are antagonize the tBHQ induced expression of luciferase in the ARE-reporter cell line AREc32.

FIG. 17 shows BTB09463 and Retinoic acid can antagonize the sulforaphane-induced expression of the ARE-driven gene AKR1C (and NQO1) at the protein level in two independent cell lines.

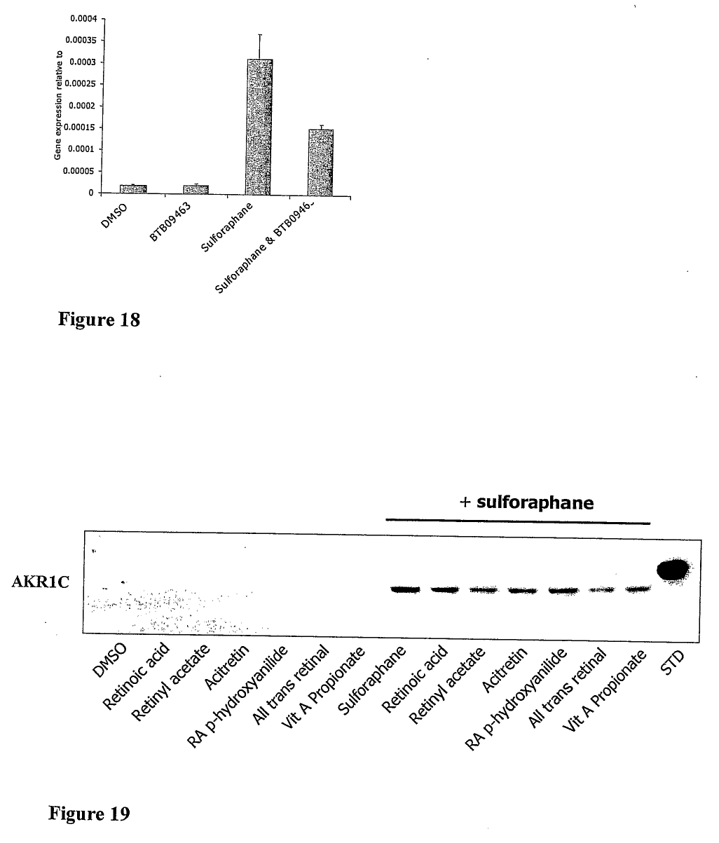

FIG. 18 shows BTB09463 can antagonize the sulforaphane induced expression of the ARE [symbol] AKR1C1 at the mRNA level.

FIG. 19 shows several retinoids can antagonize the sulforaphane induced expression of the ARE driven gene AKR1C at the protein level in MCF7 cells.

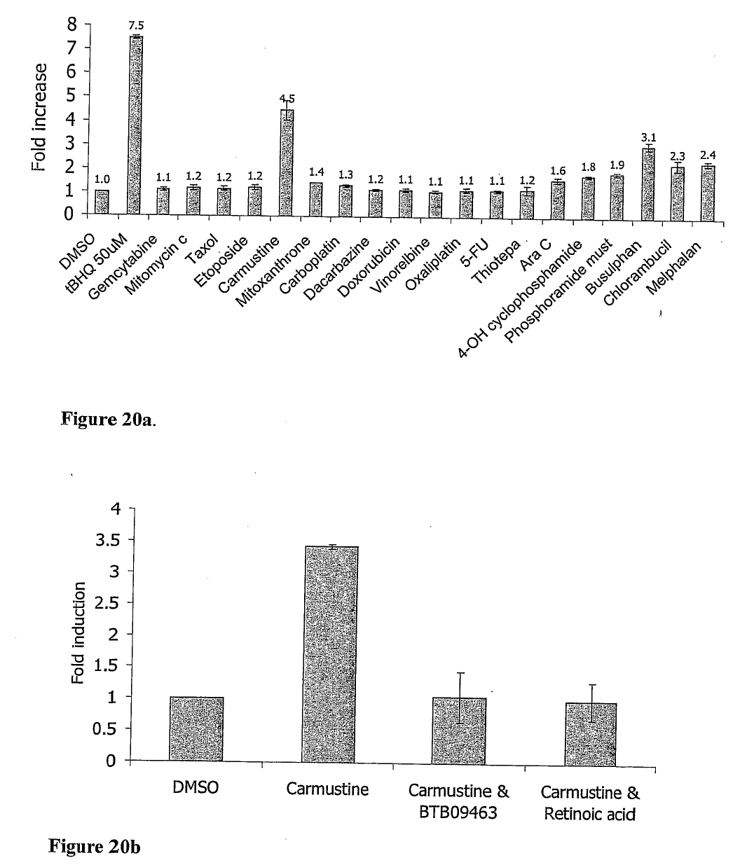

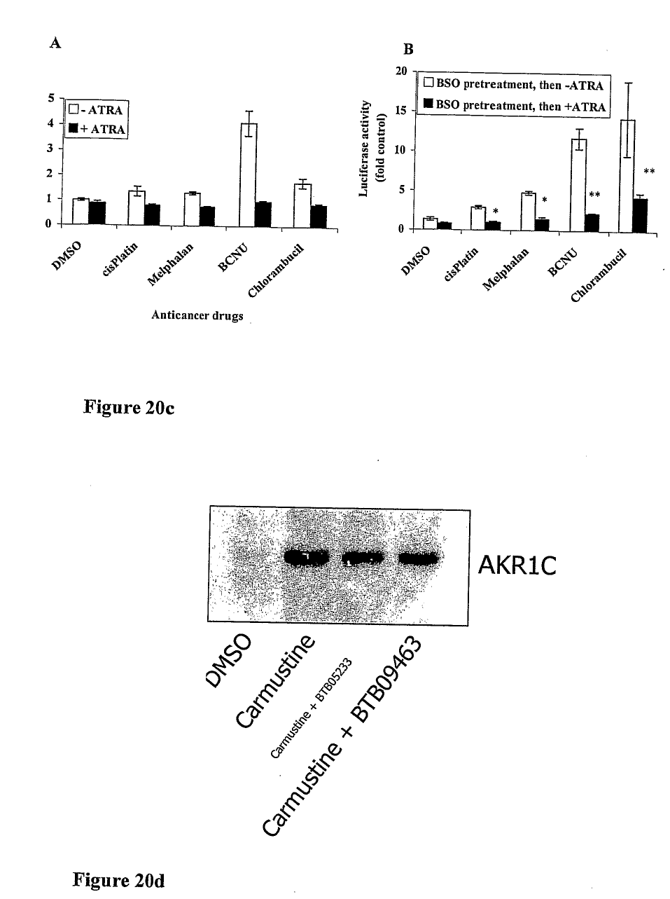

FIG. 20a shows commonly prescribed anti-cancer drugs can induce luciferase activity in the ARE reporter cell line AREc23.

FIG. 20b shows BTB09463 and Retinoic Acid antagonizes Carmustine-induced luciferase activity in the ARE reporter cell line AREc32.

FIG. 20c shows further characterisation of Retinoic Acid antagonism of chemotherapeutic agent-induced luciferase activity in the ARE reporter cell line AREc32.

FIG. 20d shows carmustine can induce ARE-gene AKR1C at the protein level in Caco-2 cells and this induction can be suppressed by concomitant treatment with BTB09463.

FIG. 20e. Further evidence to support that BTB09463 antagonizes the Carmustine induced expression of the ARE driven genes at the protein level in MCF7 cells.

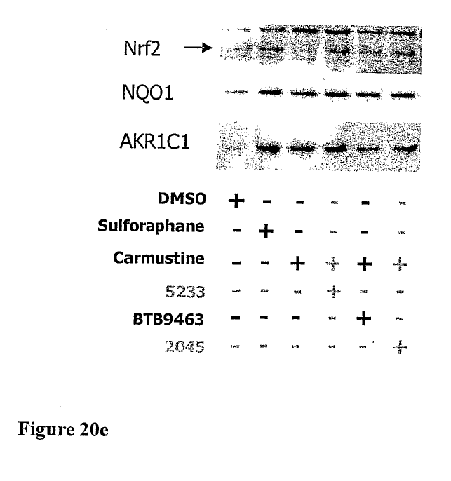

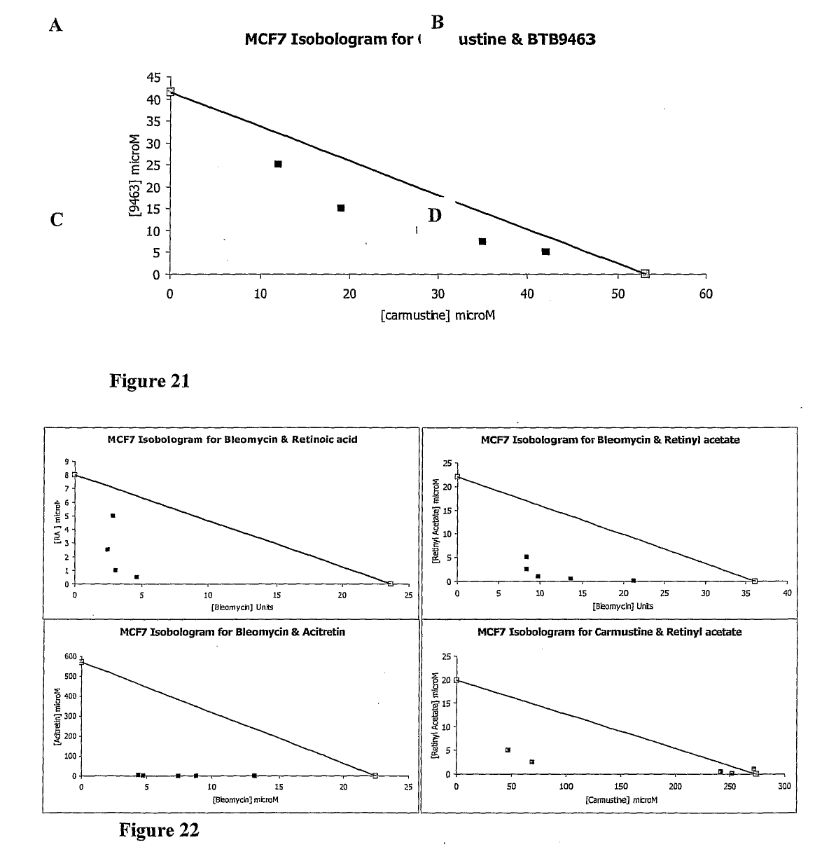

FIG. 21 shows BTB09463 increases Carmustine toxicity in MCF7 cells in a synergistic fashion.

FIG. 22 shows MCF7 cells dosed with the cytotoxic antibiotic Bleomycin (A-C) or Carmustine (D) show a massive synergistic increase in cell killing when co-treated with Retinoic acid (A), Retinyl acetate (B & D) or Acitretin (C).

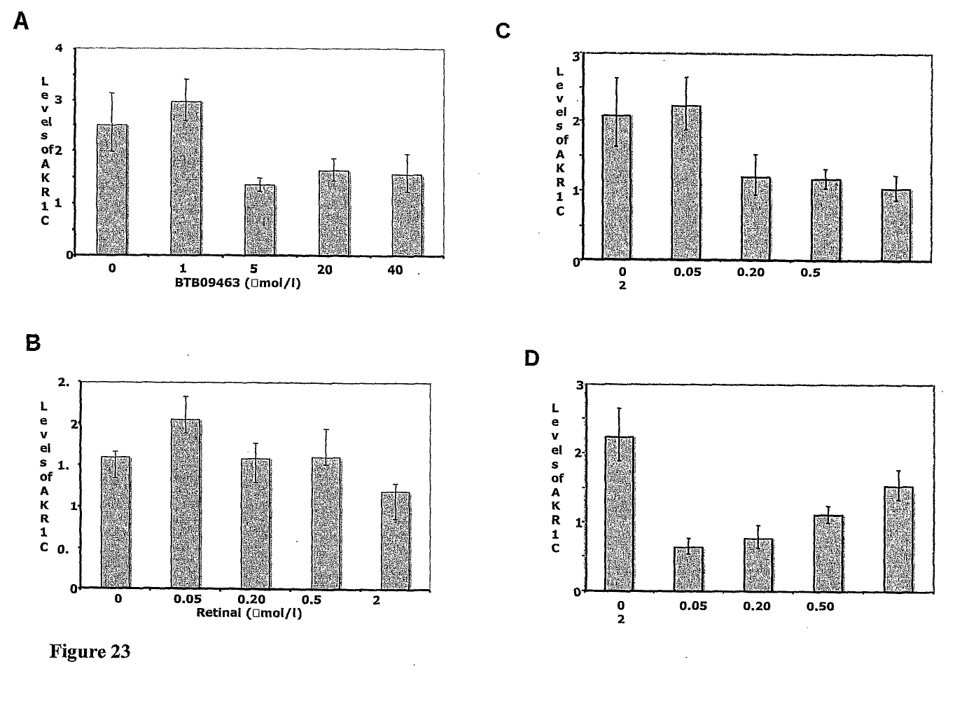

FIG. 23 shows that BTB09463 and retinoids repress the constitutive levels of endogenous AKR1C1 mRNA in A549 cells.

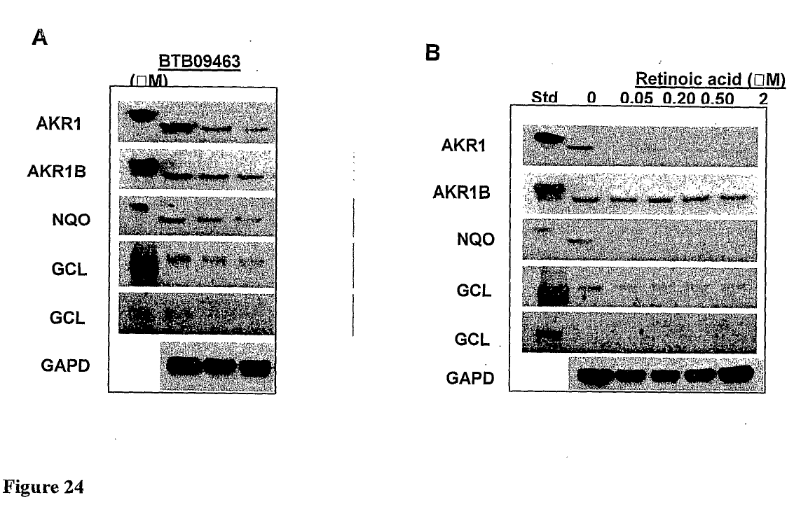

FIG. 24 shows that BTB09463 and retinoids repress levels of proteins that are members of the ARE-gene battery in A549 cells.

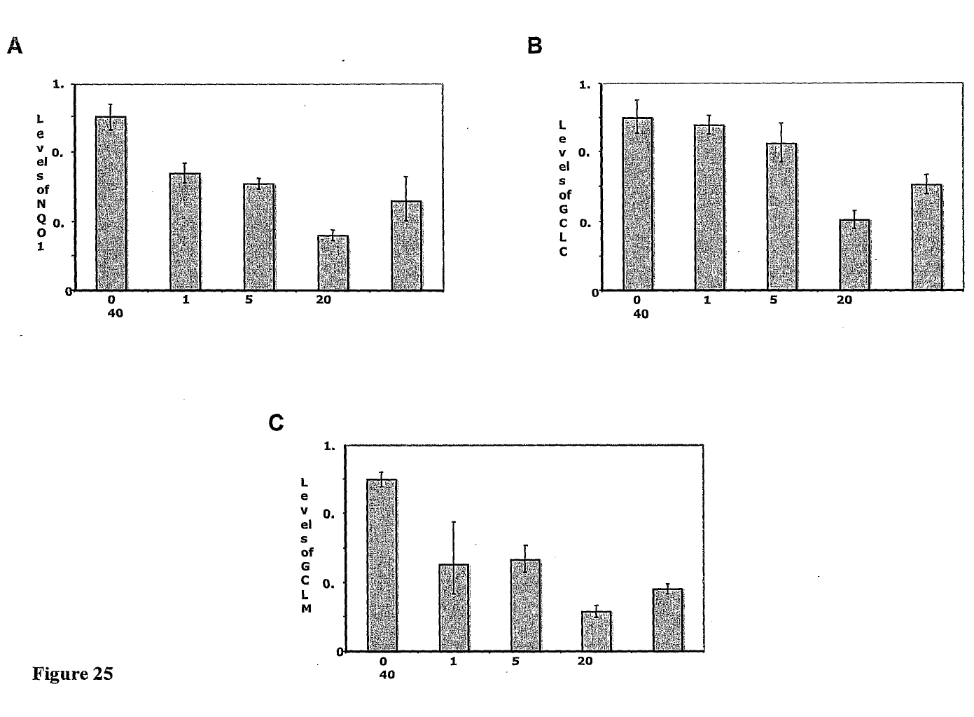

FIG. 25 shows that BTB09463 inhibits the constitutive levels of mRNA for ARE-driven genes in A549 cells.

### MATERIALS AND METHODS I

### Chemicals and Cell Culture

All chemicals unless otherwise indicated were purchased from Sigma-Aldrich Company Ltd. Dorset, UK. D.L-sulforaphane was obtained from LKT laboratories Inc. (St. Paul, Minn., USA). OTO096463 was identified from a chemical screen of a Maybridge Chemical Company Compound Library and is available from them under ACD code MFCD00173669. HepG2 (human hepatoblastoma), MCF7 (human breast carcinoma), Hepa1 (mouse hepatoma) and CHO (chinese hamster ovarian carcinoma) cell lines were obtained from the cell services of Cancer Research-UK (London, UK). The growth medium for MCF7 cells was Dulbecco's MEM with glutamax supplemented with 10% fetal bovine serum (FBS) and antibiotics. HepG2 cells were maintained in Dulbecco's MEM with glutamax supplemented with 10% FBS and antibiotics. Hepa1 cells were maintained in Dulbecco's MEM with glutamax supplemented with 10% FBS, antibiotics, 1% non-essential amino acids, and 2.5 μg/ml bovine insulin. The CHO cells were maintained in Dulbecco's MEM with glutamax supplemented with 10% FBS, antibiotics, 1% thymidine and 1% hypoxanthine. All cells were cultured at 37° C., in 95% air and 5% CO₂, and passaged every 3-4 days. All media supplements for cell culture were purchased from Life Technologies Inc. Ltd. Paisley, UK.

### Reporter Plasmids and Expression Constructs

The ARE-luciferase reporter plasmids were generated using the pGL3-promoter vector (Promega UK, Southampton, U.K.) containing an SV40 promoter upstream of the firefly luciferase gene. They are summarised in Table 1. These plasmids differ in the number of copies of ARE sequences that have been inserted, in head-to-tail orientation, through Nhe1 and Xho1 restriction sites upstream of the promoter-luc⁺ transcriptional unit. Five plasmids were made containing either one, two, four, six or eight copies of the ARE (5′-GTGACAAAGCA-3′, with the minimal functional sequence underlined) present in rat GSTA2 and mouse gsta1; these were called pGL-nxARE. A linker with the sequence of 5′-CCC3-′ and 5′-GGG3-′ on the opposite strand was placed between individual cis-elements. In addition, a plasmid named pGL-GSTA2ARE was generated that represented 41 bp of nucleotides −682 to −722 in the rat GSTA2 gene promoter (5′-GAGCTTGGAAATGGCATTGCTAATGGTGACAAAGCAACTTTG-3′, with the minimal functional enhancer shown underlined), driving the luciferase reporter gene. In mouse gsta1, this sequence is 5′-TAGCTTGGAAATGACATTGCTAATGGTGACAAAGCAACTG-3′ (Hayes & Pulford, 1995). The oligonucleotides were synthesised by MWG-BIOTECH AG (Eberserg, Germany). After the plasmids were generated, the DNA sequence of the inserts was checked.

pHyg-EF-hNrf2, a green fluorescent protein (GFP)-tagged human Nrf2 expression vector, was a gift from Prof. Masayuki Yamamoto (Institute of Basic Medical Sciences, University of Tsukuba, Japan). pEGFP-N1, a GFP expression vector employed as a negative control, was obtained from BD Clontech UK (Hampshire, UK).

### Transient Transfection and Analysis of Luciferase Reporter Gene Activity

The Dual-luciferase Reporter Assay System (Promega) was used to examine reporter gene activity in transiently transfected cells. Briefly, cells were seeded at a density of 2×10⁵ cells/well in 24-well plates and grown in the appropriate medium. After overnight incubation, the cells were transiently transfected with various ARE-luciferase reporter plasmids. The plasmid pRL-TK, encoding Renilla luciferase was used to control for transfection efficiency. Transfections were performed using Lipofectamine 2000 Reagent (Lifer Technologies Inc. Ltd., Coventry, UK) according to the manufacture's instructions. Following transfection, the culture medium was replaced 24 h later with fresh growth medium containing 50 μM tBHQ (in a solution giving a final concentration of 0.1% v/v dimethyl sulfoxide (DMSO)), which was prepared immediately before each experiment. For control experiments, vehicle alone (0.1% v/v DMSO) was added to the growth medium. Cells were left for 24 h to respond to xenobiotics before being harvested and the firefly and Renilla luciferase activities in cell lysates were measured using a luminometer (Turner Designs Model TD-20/20, Promega) following addition of Luciferase Assay Reagent II (Promega). After quenching the reaction, the Renilla luciferase reaction was initiated by adding Stop & Glo Reagent (Promega). The relative luciferase activity was calculated by normalizing firefly luciferase activity to that of Renilla luciferase.

### Generation of Stable ARE-Driven Reporter Systems

The pGL-8xARE, along with the pCDNA3.1 plasmid containing the neomycin selectable marker, was stably transfected into MCF7 cells using the calcium phosphate method (Moffat et al., 1997). Transfected cells were selected using 0.8 mg/ml G418 in the media for 3-4 weeks. The G418-resistant clones were isolated and screened by measuring their basal and inducible (by 50 μM tBHQ) luciferase activities. The firefly luciferase activity was determined as described above. Positive clones, which showed low background and high inducible luciferase activity, were passaged and maintained in the growth medium containing 0.8 mg/ml G418.

### Xenobiotic Treatments of Stable ARE-Luciferase Reporter Cells

BCNU and melphalan were dissolved in acidified ethanol as 1000× concentrated solutions. Doxorubicin, epirubicin, cyclophosphamide, methotrexate, and paclitaxol were dissolved in phosphate-buffered saline. The other anticancer agents were prepared as 1000× concentrated stock solutions in DMSO, and were stored at −20° C. until use. For treatment with anticancer drugs, cells were seeded at a density of 1.2×10⁴ cells/well in 96-well microtitre plates in growth medium. After overnight recovery, the culture medium was replaced with fresh Dulbecco's MEM supplemented with antibiotics along with the anticancer drugs of interest. An equal volume of vehicle was added to the control wells. After 24 h treatment, firefly luciferase activity was determined as described above.

Over-Expression of hNrf2 in Stable ARE-Luciferase Reporter Cells

For transfection, AREc32 cells were seeded at 1.5×10⁴ cells/well in 100 μl growth medium in 96-well plates. After overnight recovery, the cells were transfected with between 25 and 100 ng/well pHyg-EF-hNrf2 or pEGFP-N1 vectors using Lipofectamine 2000 Reagent. Following a 4 h recovery period after transfection, the culture medium was replaced with fresh Dulbecco's MEM containing glutamax and 10 μM tBHQ (or DMSO alone) supplemented with antibiotics. An equal volume of DMSO was added to the control wells. Finally, firefly luciferase activity was measured after treatment with tBHQ for 24 h.

Nrf2 siRNA Vector Preparation and Transfection

pRS hNrf2, a pSUPER RNAi vector targeting human Nrf2, was recovered from the glycerol stocks of the SUPER RNAi™ library (Netherlands Cancer Institute, Amsterdam, Netherland). The sequence of the oligo insert in the pRS-hNrf2 used in this study was 5′-GCATTGGAGTGTCAGTATG-3′, corresponding to the region from 2083 to 2101 of hNrf2 cDNA, numbering is from the A in the ATG initiation codon. A pSUPER RNAi vector targeting GFP, pRS-GFP, was also obtained from the SUPER RNAi™ library, and used as a negative control.

For transfection with pSUPER RNAi, AREc32 cells were seeded at 1.2×10⁴ cells/well in 100 μl growth medium in 96-well plates. After overnight incubation, with between 25 and 100 ng/well of the pRS-hNrf2 or pRS-GFP pSUPER vectors were transfected into the cells using Lipofectamine 2000 Reagent. Following recovery from transfection (24 h), the culture medium was replaced with fresh Dulbecco's MEM containing glutamax and 10 μM tBHQ (or DMSO alone) supplemented with antibiotics. After 24 h treatment, firefly luciferase activity was measured. The specificity of the RNAi was confirmed by TaqMan analysis.

### Statistical Analysis

Statistical comparisons were performed by unpaired Student's t tests. A value of p<0.05 was considered statistically significant.

### Results

### Generation of a Stable Cell Line Expressing a Functional ARE-Driven Reporter Trans-Gene

In this study, a series of ARE-luciferase reporter plasmids containing either one, two, four, six or eight copies of the cis-element common to the rat GSTA2 and mouse gsta1 gene promoters were made. The ARE sequences are listed in Table 1. These reporter constructs were tested by transient transfection in MCF7 and HepG2 cells. As shown in FIG. 2, increasing the number of copies of the ARE in the promoter of pGL3 had no significant effect on the basal level of luciferase activity observed under normal homeostatic conditions. However, there was a good correlation between the number of ARE copies in the pGL3 promoter vector and the level of induction of luciferase activity by tBHQ in the MCF7 cells. These results confirm the findings of Nguyen et al., 1994) in which it was demonstrated that transfection of multiple copies of the rat GSTA2-ARE increased the sensitivity of reporter gene activity (chloramphenicol acetyl transferase) to tBHQ treatment.

In order to choose an appropriate cell system for the generation of a stable reporter cell line, pGL-GSTA2.41bp-ARE was transfected into HepG2, MCF7, CHO, Hepa1 cells. As shown in Table 2, in transient transfection experiments with this construct, luciferase activity in MCF7 cells was induced up to 50-fold after an overnight treatment with 50 μM tBHQ. By contrast, the reporter gene was only induced between 2- and 4-fold following similar transfection experiments in HepG2, CHO or Hepa1 cells. Thus, our results showed that MCF7 cells expresses Nrf2 and could provide a sensitive cell system for measuring ARE-driven transcription.

We decided to employ pGL-8xARE, which contained eight tandemly arrayed copies of the minimal functional ARE, as the plasmid to generate a reporter stable cell line because this construct gave a reasonably high level of inducible luciferase production following treatment with tBHQ. To this end, pGL-8xARE and pCDNA3.1, which contained a neomycin selectable marker, were stably co-transfected into MCF7 cells and selected in the presence of G418. One hundred and fifty-three G418-resistant clones were isolated. After the first passage, thirty-two clones were kept for further monitoring according to their basal and inducible luciferase activity. Among them, one clone, defined as AREc32, showed low basal and high inducible luciferase activity, and also demonstrated a stable phenotype after more than 20 passages. The rest of the clones were discarded because they showed either a lower induction level (2- to 6-fold) by 10 μM tBHQ, or an unstable phenotype with more passages. Therefore, AREc32 cells were retained for further study.

### Induction of ARE-Driven Luciferase Activity in AREc32 Cells is Mediated by Nrf2

In order to confirm that the luciferase activity in AREc32 cells was responsive to Nrf2, this CNC bZIP protein was over-expressed in AREc32 cells by transient transfection with the expression construct pHyg-EF-hNrf2. As shown in FIG. 3, the control cells where no DNA was included in the transfection mix, gave 13-fold induction of luciferase activity when treated with 10 μM tBHQ. When 25 ng of pHyg-EF-hNrf2 plasmid DNA was used per well, neither the basal nor inducible luciferase activities were significantly affected. However, following transfection with 50 ng of pHyg-EF-hNrf2 per well, the basal level of luciferase activity increased to 2.6-fold, and the inducible level increased to 19-fold. Moreover, following transfection with 100 ng of pHyg-EF-hNrf2, the basal reporter gene activity increased to 4-fold and the inducible level to 25-fold. In different wells, the same amount of pEGFP-N1, an EGFP expression vector, was transfected into AREc32 cells as a negative control. Neither the basal nor the inducible luciferase activities were significantly affected by over-expression of EGFP.

To determine whether Nrf2 mediates induction of luciferase activity by tBHQ in AREc32 cells, an RNAi vector was used to knockdown its expression. FIG. 3B shows that transfection of AREc32 cells with either pRS-hNrf2 or pRS-GFP vectors did not affect the level of GAPDH mRNA. However, 24 h after transfection with pRS-Nrf2, the endogenous mRNA level for Nrf2 was reduced to nearly 40% of control levels, but its abundance was not affected by transfection with the pRS-GFP vector (FIG. 3B). This finding indicates that transfection of pRS-hNrf2 specifically suppressed expression of the bZIP factor.

Transfection of AREc32 cells with pRS-hNrf2 reduced the basal level of luciferase activity to 60% of control levels (FIG. 3C). When 25 ng of pRS-hNrf2 DNA was used per well, the inducibility of luciferase activity was not affected significantly, compared to the control cells (10-fold induction) where no DNA was included in the transfection mix. When 50 ng of pRS-hNrf2 DNA was used per well, induction of luciferase activity by 10 μM tBHQ was reduced to 8-fold. When 100 ng of pRS-hNrf2 DNA was used per well, only 6-fold induction by tBHQ was detected. In different wells, the basal and inducible luciferase activity was not affected when AREc32 cells were transfected with the same amount of pRS-GFP DNA, which targeted GFP mRNA (FIG. 3C). These data indicate both basal and inducible luciferase activities in AREc32 cells are mediated by Nrf2 through the ARE.

### Time- and Dose-Dependent Induction of Luciferase in AREc32 Cells

Luciferase activity in AREc32 cells could be induced by in a time- and dose-dependent manner; after treatment for 24 h, luciferase activity was increased 2-fold by 1 μM tBHQ, and 5-fold by 5 μM tBHQ (see FIG. 4A and Table 3). A maximum luciferase activity (around 10-fold increase) was seen following treatment with 10 μM tBHQ. Induction of luciferase activity by tBHQ was also time-dependent; it increased 4-fold after 8 h treatment with 10 μM tBHQ, and reached 10-fold 18 h after treatment with the same dose of tBHQ. A similar magnitude of induction of luciferase activity in AREc32 cells was observed after 24 h exposure to 10 μM sulforaphane (SUL), a potent NQO1 and AKR1C enzyme inducer (Bonnesen et al., 2001).

### The Effect of Anticancer Drugs on ARE-Reporter Gene Expression

In order to find out whether cancer chemotherapeutic agents modulate the Nrf2-ARE system, a number of anticancer drugs were screened using AREc32 cells. Based on the IC₅₀ results (data not shown), AREc32 cells were treated for 24 h with multiple sub-lethal doses of the therapeutic agents. According to their effect on luciferase activity, these drugs were divided in Table 4 into three groups: no significant effect, modest activators, and strong activators. Thus, doxorubicin, epirubicin, paclitaxol (taxol), methotrexate and thiotepa treatment had no effect on the level of luciferase activity in AREc32 cells. The alkylating agents cisplatin, mephalan and the redox-cycling compound etopside modestly increased luciferase activity. Treatment of AREc32 cells with alkylating agents chlorambucil, mitozantrone and BCNU, elicited a stronger induction of luciferase activity that was between 2- and 4-fold.

Using AREc32 cells we found that cyclophosphamide treatment did not have any effect on ARE-luciferase activity. By contrast, its major metabolite acrolein was found to be a potent ARE activator; 10 μM acrolein gave a 27-fold increase in luciferase activity.

### Activation of ARE-Driven Gene Expression by Anticancer Drugs is Redox Dependent

In order to examine the whether cellular GSH level has any effect on the ability of anticancer drugs to activate luciferase activity, we pretreated AREc32 cells with 50 μM BSO for 24 h before challenging them with chemotherapeutic agents. As can be seen in FIG. 5A, the pre-treatment with BSO caused the induction of luciferase activity by cisplatin and melphalan to be increased to 3- and 5-fold, respectively. More remarkably, BSO caused the induction of luciferase activity by chlorambucil and BCNU to be increased to >10-fold. Such inductions were nearly completely repressed by the addition of 5 mM NAC (FIG. 5A). For the treatments of etopside and mitozantrone, we found that BSO pre-treatment did not change luciferase activity significantly (data not shown).

To find out whether anticancer drugs similarly activate the expression of an endogenous Nrf2-regulated gene, we examined expression of AKR1C in AREc32 cells. Without pre-treatment with BSO, the mRNA level of AKR1C was only slightly increased by the treatment of melphalan, cisplatin, chlorambucil. However, when the cells were pre-treated with 50 μM BSO for 24 h, melphalan and cisplatin increased the expression of AKR1C mRNA by 3- and 4-fold, respectively, and chlorambucil increased this mRNA 31-fold (FIG. 5B). Treatment with BCNU induced the expression of AKR1C mRNA 3-fold, and with pre-treatment of BSO BCNU induced AKR1C mRNA 42-fold (FIG. 5B). Immunoblotting revealed that AKR1C protein was also increased by these anticancer drugs (FIG. 5C). BSO pre-treatment did not further enhance the expression of AKR1C protein by tBHQ treatment. However, this is possibly because the induction of AKR1C by 10 μM tBHQ alone has already reached the maximum level.

### Discussion

We have generated a stable ARE-reporter human mammary cell line, AREc32, derived from MCF7 cells, in which only the minimal enhancer sequence is present to direct expression of the luciferase trans-gene. The ARE employed for this purpose was designed around that found in the promoters of both rat GSTA2 and mouse gsta1. In the case gsta1, its basal and inducible expression has been shown to be regulated by Nrf2 in vivo (Chanas et al., 2002). We also used the ARE from the promoters of GSTA2 and gsta1 because, unlike that in human NQO1, it does not contain an embedded AP1 site and the absence of this site within the ARE should facilitate interpretation of induction of reporter gene activity. We have shown that in the AREc32 cells expression of luciferase activity was mediated by Nrf2 and was sensitive to redox status. This cell line gave a 10-fold induction of reporter activity by 10 μM tBHQ, and therefore provides a good model system that can be used to screen chemical libraries in order to identify agonists and antagonists of Nrf2.

Response to AREc32 cells to anti-cancer agents.

In our study, we used AREc32 cells to examine the ability of anticancer alkylating agents, to induce ARE-driven gene expression. We found that the cisplatin, etoposide (VP16), mitozantrone, melphalan, chlorambucil and BCNU were capable of inducing luciferase. Induction of ARE-luciferase by these chemotherapeutic agents was found to be redox-sensitive, insofar as it was augmented by BSO pre-treatment and suppressed by NAC (FIG. 5A). Interestingly, this suggests that sub-optimal treatment of patients with certain anticancer drugs may induce cytoprotective defences in tumours that are controlled by Nrf2. Furthermore, the redox status of cells in the tumour will influence their ability to activate such defences.

### MATERIALS AND METHODS II

### Chemicals

Retinoids used in the treatment of AREc32 cells, were prepared in DMSO, and that administered to mice, were prepared in corn oil. Retinoid solutions were stored at −70° C. in aliquots, and only used once after each was thawed. The experimental procedures involved the handing of retinoids were performed in subdued light.

### Animals

Homozygous Nrf2 KO mice and mouse genotyping were as described previously (Itoh et al., 1997). Two month old, C57BL/6 nrf2^{-/-} and nrf2^{+/+} male mice were used in this study. Animals were maintained in a 12-h light-dark cycle, with free access to food and water. The mice were weighed daily during the experiment period. All animal procedures were carried out under UK Home Office license and after gaining local ethical committee approval.

Two feeding experiments were carried out. In Experiment 1, at the first stage, which lasted for four weeks, Nrf2 (+/+) mice were maintained on a retinoic acid deficient VAD diet (Special Diet Services, Witham, Essex, UK). At the second stage, lasted for two weeks, the mice were divided into three experiment groups, and their diets and treatments are as follow: (a) group 1, VAD diet; (b) group 2, VAD diet, and that ATRA was administered daily at a dose of 10 mg/kg BW; (c) group 3, VAD diet, and that corn oil was administered intraperitoneally daily. In experiment 2, Nrf2 (−/−) mice were maintained on control or VAD diet for six weeks.

By the end of six weeks, mice were sacrificed and their small intestines immediately excised, frozen in liquid nitrogen, and kept at −70° C. until use. The feeding experiments were repeated three times and each experiment group contained two or three animals.

### Cell Culture and the Measurement of Luciferase Activity

AREc32 cells were prepared as described in the above Materials & Methods Section and were maintained in the growth medium (Dulbecco's MEM with glutamax supplemented with 10% fetal bovine serum (FBS) and antibiotics) containing 0.8 mg/ml G418, at 37° C., in 95% air and 5% CO₂, and passaged every 3-4 days. The media supplements for cell culture were purchased from Life Technologies Inc. Ltd. (Paisley, UK).

For xenobiotic treatment, AREc32 cells were seeded in a 96-well plate at 1.2×10⁴ cells/well in the growth medium. After 24 h recovery, the culture medium was replaced with fresh DMEM supplemented with antibiotics containing xenobiotics (0.1% v/v). Cells were left for 24 h to respond to xenobiotics before being harvested and the firefly luciferase activities in cell lysates were measured using a luminometer (Turner Designs Model TD-20/20, Promega) following addition of Luciferase Assay Reagent (Promega). For control experiments, vehicle alone (0.1% v/v DMSO) was added to the medium.

### Real-Time Quantitative PCR (RT-PCR)

Total RNA was isolated with TRizol and further purified with RNeasy Mini Kit (Qiagen Ltd) in accordance with the manufacturer's instructions. The A260/A280 ratio of total RNA used was typically ≧1.9. The quality of RNA was assessed using the Agilent 2100 Bioanalyzer. RT-PCR was performed as described previously (Wang et al., 2005). The primers were synthesised by MWG-BIOTECH AG. The probes, which were labelled with a 5′ fluorescent reporter dye (6-carboxyfluorescein) and a 3′ quenching dye (6-carboxytetramethylrhodamine), were synthesised by Qiagen Ltd. (Germany). Each assay was performed in triplicate. The specificity of PCR amplifications from the various sets of oligonucleotide primers was examined routinely by agarose-gel electrophoresis. The results were analysed by using 7700 system software. The level of 18S rRNA was used as an internal standard. The sequences for the primers and probes for measuring cDNA corresponding to human AKR1C mRNAs have been described previously (Devling et al., 2005).

### Western Blot Analysis

Whole-cell extracts were prepared from the cultured cells as described previously (Wang X J 2006). Briefly, the cells were lysed in an extraction buffer containing 0.1 M Hepes pH 7.4, 0.5 M KCl, 5 mM MgCl₂, 0.5 mM EDTA, 20% glycerol supplemented with protease inhibitor mixture (Roche Diagnostics). Protein samples (30 μg) were separated on SDS-PAGE gels using a standard protocol. Immunoblotting was carried out using antiserum raised against AKR1C as described previously (O'Connor et al., 1999). Intestinal cytosol was prepared as described previously (McMahon et al., 2001). 5 μg of protein from the intestinal sample was routinely separated by SDS-PAGE. Western immumoblotting was performed to estimate the levels of NQO1 and GSTs proteins. The sources of these primary antibodies used have been described previously (Hayes et al., 2000; Kelly et al., 2000). In all cases, immunoblotting with antibody against actin (Sigma) was performed to confirm equal loading.

### Electrophoretic Mobility Shift Assays (EMSA)

The nuclear extracts used for EMSA were prepared according to a procedure described elsewhere (Moffat et al., 1997). Double-stranded DNA probes (ARE, 5′-GAGCTTGGAAATGGCATTGCTAATGGTGACAAAGCAACTTTG-3′ [core sequences are underlined]) end labeled with [γ-³²P]ATP and T₄ polynucleotide kinase were used for gel shift analyses, as previously described (Moffat et al., 1997). In some analyses, specificity of binding was determined by competition experiments, which were carried out by adding a 200-fold molar excess of an unlabeled oligonucleotide to the reaction mixture before the labeled probe was added. Samples were separated in 4% polyacrylamide gels at 100 V. The gels were dried, and subjected to autoradiography.

### Biotinylated ARE Oligonucleotide Pull-Down Assay

Nuclear extracts used for the pull-down assay were prepared as described previously (Deng et al., 2003). Briefly, AREc32 cells were lysed in two packed cell volumes of buffer A containing 10 mM Hepes, pH 8.0, 1.5 mM MgCl₂, 200 mM sucrose, 0.5% Nonidet P-40, 10 mM KCl, 0.5 mM dithiothreitol, 0.1 mM sodium orthovanadate, 1 mM EGTA supplemented with protease inhibitor mixture (Roche Diagnostics) for 5 min at 4° C. The crude nuclei were collected by microcentrifugation, and resuspended in three packed cell volumes of buffer B (PBS, pH. 7.4, 1.0 mM EDTA, 1.0 mM dithiothreitol plus protease and phosphatase inhibitors in buffer A). Nuclei were then disrupted by sonication at 4° C., followed by by microcentifugation to remove the debris. The supernatant containing nuclear extract proteins was collected and stored at −70° C.

Double stranded 5′-biotinylated ARE probe, represented 42bp of nucleotides −682 to −722 in the rat GSTA2 gene promoter, was synthesized by MWG-BIOTECH AG. Its sequence is 5′-GAGCTTGGAAATGGCATTGCTAATGGTGACAAAGCAACTTTG-3′. In addition, a nonrelevant biotinylated probe (mock), 5′-AGAGTGGTCACTACCCCCTCTG-3′, was also synthesized to serve as a negative probe control.

The ARE-pull down assay was carried out as described previously (Deng et al., 2003). Briefly, 720 nM 5′-biotinylated ARE probe was mixed with 500 μg of nuclear extracts from AREc32 cells treated with different compounds and 100 μl of 4% streptavidin-agarose beads (Sigma). The final volume was adjusted to 500 μl with nuclear extract buffer B. The mixture was rocked at room temperature for 1 h, and the tube was centrifuged at 5000 g for 30 s. The pellet was washed four times with iced PBS and the pulled down mixture was analysed on SDS-PAGE. Nrf2 proteins were identified by immunoblotting using rabbit polyclonal Nrf2 antibody.

### Statistical Analysis

Statistical comparisons were performed by unpaired Student's t tests. A value of p<0.05 was considered statistically significant.

### Results II

### Antagonism of Inducible ARE-Driven Gene Expression by All Trans-Retinoic Acid

The MCF7-ARE reporter cell line was treated with a number of compounds known to activate the ARE including tBHQ, acrolein, β-naphthoflavone (NF) and Sul. As expected, all of these inducing agents increased luciferase activity in AREc32 cells (FIG. 6). Treatment of AREc32 cells with tBHQ, acrolein, NF and Sul in the presence of 1 μM ATRA however significantly attenuated the increase in ARE-driven luciferase activity affected by the inducing agents. Indeed, following subtraction of the DMSO control from the values obtained, there was almost complete ablation of luciferase activity. In a subsequent experiment (shown in FIG. 7A) we examined the dependence of inhibition of the ARE-driven response on retinoic acid concentration and also the ability of other retinoid derivates to inhibit the ARE response. Interestingly, all 3 retinoids inhibited the ARE response in a similar dose-dependent manner, the IC₅₀ values being approximately 3×10⁻⁷M. It is known that these three retinoid derivatives all bind with approximately equal potency to the retinoic acid receptor suggesting that this mediates the responses observed. In addition, the time dependence of the inhibition of luciferase activity by retinoic acid was determined. As shown in FIG. 7B, after a lag phase of approximately 3 hour, luciferase activity in tBHQ-treated cells increased almost linearly over a 24-hour period. However, when AREc32 cells were treated simultaneously with tBHQ and ATRA, the lag phase increased from 3 hour to 16 hours, and thereafter only a modest increase in luciferase activity was between 16 and 24 hours.

All Trans-Retinoic Acid Prevents Induction of Endogenous Genes by tBHQ

In order to establish whether retinoic acid could inhibit the expression of endogenous genes regulated through the ARE, we investigated the effects of ATRA on the induction of the AKR1C1 gene by tBHQ (FIG. 8A). In this experiment tBHQ induced the expression of AKR1C1 mRNA by approximately 15-fold and this induction was markedly repressed (to just 3-fold induction) by co-incubation with retinoic acid. After subtracting the DMSO control, the inhibition was estimated to be approximately 85%. We then investigated the effect of ATRA on the induction of AKR1C protein by Western Blot analysis. As can be seen in FIG. 8B, the level of this protein was also markedly reduced. Scanning of the Western blots indicated that this reduction was approximately 50%; this apparent discrepancy between the TaqMan and immunoblotting data is probably due to a lack of specificity in the antibody raised against AKR1C1 as it will cross-react with AKR1C2 and probably AKR1C3.

In order to investigate whether the observations in MCF7 cells could also be extrapolated to the expression of ARE-regulated genes in vivo, we carried out an experiment where mice were fed a retinoic acid-deficient (i.e. vitamin A-deficient, VAD) diet. Interestingly, in wild-type mice placed on a vitamin A-deficient diet for 6 weeks, a profound induction of the ARE-regulated genes GstM5 GCLC, NQO1 and GstA1 was observed (FIG. 9). The induction of these genes by the VAD diet was dependent on Nrf2 as no increase in GstM5 GCLC, NQO1 and GstA1 was observed in nrf2^{-/-} mice. On daily administration of ATRA to wild-type mice during the last 2 weeks of them being placed on the VAD diet, the induction of ARE-driven genes was almost completely reversed in the small intestine. This finding demonstrates that the repressive effects of retinoic acid are relevant to the in vivo situation in the GI tract.

### All Trans-Retinoic Acid Prevents Induction of ARE-Driven Gene Expression by Anti-Cancer Drugs

Further experiments were performed to determine whether retinoic acid can inhibit the induction of Nrf2-regulated genes by a series of anticancer drugs (FIG. 10). Of the anticancer drugs, cisplatin, melphalan and chlorambucil were weak inducers of ARE-driven gene expression (Table 4). By comparison, BCNU was a stronger inducing agent. Induction of ARE-driven luciferase activity by each of these anticancer drugs was prevented by inclusion in the media of ATRA along with the chemotherapeutic agents (FIG. 10A). Induction of luciferase activity by these agents could be markedly enhanced by pre-treating the AREc32 cells for 24 hours with the glutathione depleting agent L-buthionine-S,R-sulfoximine (BSO) and, indeed, under these conditions all of the anticancer drugs used were efficient inducers of the ARE reporter; BCNU and chlorambucil inducing between 10-15-fold. In all of these experiments, ATRA was a potent inhibitor of the induction of ARE. This was particularly the case for experiments where cells were pre-treated with 50 μM BSO where ARE responses were reduced almost to background levels following subtraction of the DMSO control values. These data demonstrate that retinoic acid has the capacity to attenuate an ARE response induced by currently used anti-tumour agents.

### All Trans-Retinoic Acid Does Not Influence the Stability of Nrf2

In order establish the mechanism by which retinoic acid exerts its inhibitory effects, we investigated whether the nuclear concentration of Nrf2 was changed in the presence of this compound. This, however, was found not to be the case (FIG. 11). We therefore conclude that ATRA does not antagonise Nrf2-mediated induction of gene expression by either destabilizing the bZIP factor or by preventing its nuclear translocation.

In order to establish whether retinoic acid inhibited the binding of Nrf2 to its enhancer, we carried out electrophoretic mobility shift assays using a core ARE binding sequence. Three complexes were observed to interact with this enhancer (FIG. 12) and their binding was reduced in the presence of tBHQ and retinoic acid, indicating that retinoic acid does interfere with the activation of the ARE enhancer element (track 4 v. track 2). Using a further method for the loading of Nrf2 on the ARE enhancer, we were able to confirm that retinoic acid inhibited the binding of Nrf2 to the ARE in the presence of tBHQ (FIG. 13). We therefore conclude that ATRA inhibits the ability of Nrf2 to transactivate gene expression by interfering with its recruitment onto AREs in gene promoters.

### Discussion II

The data described above show that retinoic acid and its various derivatives antagonise induction of ARE-driven gene expression by model inducing agents. Furthermore, this antagonism of ARE-driven gene expression requires relatively low doses (i.e. 10-7 M) of ATRA suggesting retinoids are potent inhibitors of Nrf2 activity. The finding that ATRA also blocks induction of ARE-driven genes by anticancer drugs suggests retinoids will prevent tumours from switching on cytoprotective genes in response to chemotherapy. Thus, retinoids may allow anticancer drugs to be more therapeutically effective if they are co-administered with the agent.

### FURTHER EXAMPLES

Further experiments were conducted and their results are shown in FIGS. 14-25. The methods and results for each experiment are described below:

Method: AREc32 cells were seeded out in 96 well plates and treated with DMSO (control), tBHQ (50 μM), tBHQ+BTB09463 (5 μM) or tBHQ+Retinoic acid (1 μM). After 24 hours incubation, cells were washed and lysed before measuring luciferase activity. BTB09463 is 1-{4-[(3,4-dichlorobenzyl)oxy]phenyl}ethan-1-one.

Results: Luciferase activity is highly inducible by tBHQ in the AREc32 reporter cell line, in this experiment showing a 14-fold induction of expression as compared to the DMSO control. Co-treatment with BTB09463 or Retinoic acid markedly suppressed this induction, by approximately 65% and 75% respectively. See FIG. 14.

Method: AREc32 cells were seeded out in 96 well plates and dosed with BTB09463 (2.5, 5 or 10 μM) for 0, 24 or 48 hrs before treatment with tBHQ (50 μM). 24 hours after the addition of tBHQ, cells were washed and lysed before measuring luciferase activity.

Results: Suppression of tBHQ-mediated induction of luciferase expression was identical under each dosing regimen. See FIG. 15.

Method: ARE c32 cells were seeded out in 96 well plates and treated with DMSO (control), tBHQ (50 μM), tBHQ+retinoid (0.25, 0.5 and 1 μM). After 24 hours incubation, cells were washed and lysed before measuring luciferase activity.

Results: All retinoids tested were capable of down-regulating the tBHQ induced luciferase expression in the ARE-reporter cell line ARE c32. See FIG. 16

Method: A. Caco-2 cells were treated with the known ARE-gene inducer sulforaphane (5 ρM), either alone or concomitantly with BTB09463 (5 μM) or Retinoic acid (1 μM). After 24 hrs, cell lysates were prepared and Western blotting performed to measure the levels of AKR1C protein. B. MCF7 cells were treated with sulforaphane (5 μM), either alone or concomitantly with BTB09463 (5 μM). After 24 hrs, cell lysates were prepared and Western blots performed to detect the levels of AKR1C and NQO1; GAPDH was used as a loading control in MCF7 cells.

Results: A. BTB09463 and Retinoic acid dramatically reduced the ability of sulforaphane to induce AKR1C in the colon cancer cell line (Caco-2). B. BTB09463 potently inhibited the sulforaphane-driven induction of ARE genes NQO1 and AKR1C in the breast cancer cell line MCF7. See FIG. 17.

Method: Caco-2 cells were treated with DMSO (control), BTB09463 (5 μM), sulforaphane (5 μM) or a combination of sulforaphane plus BTB09463. After 24 hrs treatment, cells were harvested and RNA isolated. cDNA for each sample was generated by reverse transcription and subsequently used in real time PCR analysis of gene transcription (TaqMan analysis) for the ARE-driven genes AKR1C1 and NQO1. Data was normalised to the internal control 18S RNA and the relative levels of AKR1C1 and NQO1 calculated using the comparative CT method.

Results: Sulforaphane induced a 12-fold induction of AKR1C1 mRNA which was strongly inhibited by co-treatment with BTB09463 (50% reduction). NQO1 was less markedly induced by sulforaphane, however in this case mRNA expression was reduced to basal levels when co-treated with BTB09463. See FIG. 18.

Method: MCF7 cells were treated with the known ARE-gene inducer sulforaphane (5 μM), either alone or with various retinoids (0.5 μM). After 24 hrs, cell lysates were prepared and Western blots were performed to detect the levels of AKR1C protein present.

Results: Retinyl acetate, acitretin, all-trans retinal and vitamin A propionate all reduced the expression of sulforaphane-induced AKR1C in MCF7 cells. See FIG. 19

Method: ARE reporter cell line AREc23 was seeded into 96 well plates and treated with a previously determined non-toxic concentration of cytotoxic drug. After 24 hours incubation, cells were washed and lysed before measuring luciferase activity.

Results: The majority of drugs tested exhibited modest induction of ARE-driven luciferase activity, typically ranging from 10-60% induction. Amongst the chemotherapeutic drugs, alkylating agents proved to be the strongest inducers of luciferase activity, with busulphan (3.1-fold induction) and carmustine (BiCNU) (4.5-fold induction) being the most potent. See FIG. 20a

Method: ARE reporter cell line AREc23 was seeded into 96 well plates and treated with DMSO (control), Carmustine (100 μM), Carmustine+BTB09463 (5 μM) or Carmustine+Retinoic acid (5 μM) After 24 hours incubation, cells were washed and lysed before measuring luciferase activity.

Results: BTB09463 and Retinoic acid can both completely suppress the carmustine-mediated induction of luciferase activity in the ARE-reporter cell line (AREc32). See FIG. 20b.

Method: A. ARE reporter cell line AREc23 was seeded into 96 well plates and treated with DMSO (control), Alkylating agents alone, Alkylating agents+Retinoic acid (ATRA). After 24 hours incubation, cells were washed and lysed before measuring luciferase activity. B. Modified repeat of experiment A, with cells being pretreated with L-buthionine-(SR)-sulfoximine (BSO), an inhibitor of enzymes in the glutathione synthesis pathway. After 24 hours incubation, cells were washed and lysed before measuring luciferase activity.

Results: A. Retinoic acid completely ablates the chemotherapeutic agent-mediated induction of luciferase activity in the ARE-reporter cell line (AREc32). B. Pretreatment of AREc32 cells with BSO caused a marked increase in the level of chemotherapeutic agent-mediated luciferase activity. Retinoic acid was still capable of significantly antagonising this increased response. See FIG. 20c.

Method: Caco-2 cells were treated for 24 hrs with DMSO (control), Carmustine (100 μM), Carmustine+BTB09463 (5 μM). After 24 hrs, cell lysates were prepared and Western blots to detect the levels of AKR1C.

Results: Carmustine treatment of Caco-2 cells caused massive induction of AKR1C protein expression, which was attenuated by co-administration of BTB09463. This result also reproduced in LS 174 cells (data not shown). See FIG. 20d.

Method: MCF7 cells were treated for 24 hrs with DMSO (control; Lane 1), Sulforaphane (5 μM) (Lane 2), Carmustine (100 μM) (Lane 3), Carmustine+BTB09463 (5 μM) (Lane 5). (Lane 4 represents experimental conditions irrelevant to the application). After 24 hrs, cell lysates were prepared and Western blots to detect the levels of Nrf2, NQO1 and AKR1C proteins.

Results: Carmustine treatment caused over-expression of NQO1 and AKR1C protein. Over expression of ACR1C and NQO1 protein was attenuated by co-administration of BTB09463. See FIG. 20e.

Method: To generate the data needed for an Isobologram analysis, cytotoxicity assays using MCF7 cells were performed to determine the LD₅₀ of carmustine alone, BTB09463 alone, and carmustine in the presence of a range of fixed concentrations of BTB09463. Assays were performed in 96 well plates with an incubation time of 72 hrs. Cell toxicity was determined using an ATP chemiluminescent assay.

Results: Data points which lie under the line plotted between the LD₅₀ of the two individual compounds being tested, alone indicate combinations which exhibit synergistic cytotoxic behaviour, the further away from the line, then the more synergistic the relationship is. By this definition there is a modest synergy between carmustine and BTB09463. See FIG. 21.

Method: Assays were carried out essentially as described for FIG. 21.

Results: Data indicated that there is a very potent, synergistic increase in cell killing when MCF7 cells are co-treated with Bleomycin and Retinoic acid, Retinyl acetate or Acitretin. Synergy was also observed for certain combinations of Carmustine and Retinyl acetate, with marked increase in potency at lower Carmustine concentrations. See FIG. 22.

Method: A549 cells were treated with DMSO (control), BTB09463 (1, 5, 20, 40 μmol/l), or retinoids (0.050, 0.20, 0.50, 2.0 μmol/l). After 24 hrs, total RNA was prepared and Taqman analysis was performed to detect the levels of mRNA for AKR1C1.

Results: The Taqman results showed BTB09463, all-trans retinoic acid, all-trans retinal, and retinyl acetate all inhibited the constitutive expression of AKR1C1 in a concentration-dependent manner. See FIG. 23

Method: A549 cells were treated with DMSO (control), BTB09463 (1, 5 μmol/l), or retinoic acid (0.050, 0.20, 0.50, 2.0 μmol/l). After 24 hrs, cell lysates were prepared and Western blotting was performed to detect the protein levels of ARE-driven genes (AKR1C1, AKR1B10, NQO1, GCLC, GCLM).

Results: The results of the Western blots analyses showed BTB09463 and all-trans retinoic acid repress the constitutive levels of AKR1C1, AKR1B10, NQO1, GCLC and GCLM. In all the proteins examined the repression by BTB09463 and retinoic acid was at least 50% relative to levels seen in the control. See FIG. 24.

Method: A549 cells were treated with DMSO (control), BTB09463 (1, 5, 20, 40 μmol/l), or retinoic acid (0.050, 0.20, 0.50, 2.0 μmol/l). After 24 hrs, total RNA was prepared and Taqman analyses were performed to detect the mRNA levels of the endogenous ARE-driven genes NQO1, GCLC, GCLM.

Results: The results of the Taqman analyses showed BTB09463 and all-trans retinoic acid repressed the constitutive mRNA levels of NQO1, GCLC and GCLM in a concentration-dependent manner. See FIG. 25.

Method: MCF-7 or A549 cells were seeded into 96 well plates. After 24 h the cells were treated with either Carmustine or Bleomycin alone or in the presence of BTB09463 (5 and 20 μmol/l for MCF-7 and A549 cells, respectively) for 72 h. Cells were washed and then lysed to determine their ATP levels to determine their viability.

Results: Combinations of cytotoxic cancer drugs with either BTB09463 or retinoic acid was found to be more cytotoxic than the drug treatments alone. This has resulted in the lowering of the IC₅₀ values for carmustine and bleomycin by greater than 50%. See Table 5.

In summary the Nrf2 transcription factor confers protection against agents that cause oxidative stress and chemicals that are electrophiles because it controls the expression of a battery of genes encoding antioxidant enzymes, drug-metabolising enzymes, drug efflux pumps, heat shock proteins and chaperones, as well as anti-inflammatory proteins. The genes that Nrf2 controls all contain an antioxidant response element (ARE) in their promoters. Nrf2 activity and the levels of proteins it regulates are increased in pre-neoplastic lesions and in many tumours, presumably contributing to survival of pre-malignant and malignant cells. In this invention we describe retinoids and other small molecule inhibitors (SMIs, e.g. BTB09463) that antagonise Nrf2 activity and increase the cytotoxic effects of cancer chemotherapeutic agents. In a human mammary MCF7-derived stable reporter cell line, the retinoids and other SMIs antagonise the induction of the ARE-driven luciferase reporter gene by tert-butylhydroquinone (tBHQ) and sulforaphane (Sul), compounds that are known to activate Nrf2 by preventing Keap1-mediated degradation of the factor. The retinoids and other SMIs also antagonise the induction of endogenous ARE-driven genes such as aldo-keto reductase (AKR) 1C1, NAD(P)H:quinone oxidoreductase 1 (NQO1), and the glutamate cysteine ligase catalytic (GCLC) and modifier (GCLM) subunits, at both the mRNA and the protein level, in various lines including the human mammary MCF7 and MDA157 cells, and the human colon LS174 and Caco2 cells. Certain cancer chemotherapeutic agents (e.g. Chlorambucil, Carmustine, Melphalan, Busulphan, Cisplatin) induce ARE-driven genes, suggesting that they can stimulate an adaptive response that induces resistance against the drug and, as was the case with tBHQ and Sul, this induction can similarly be antagonised by retinoids and the other SMIs. In the A549 non-small cell lung carcinoma cell line, which possesses constitutively active Nrf2 (because of loss of negative regulation by Keap1) retinoic acid and the SMIs reduce the extent to which AKR1C1, NQO1 and GCLC are over-expressed. The ability of retinoids to inhibit the activity of Nrf2, and thus the expression of the genes it regulates, is mediated by the retinoic acid receptor alpha (RARα). Co-immunoprecipitation experiments have shown that inhibition of ARE-driven gene expression by retinoic acid occurs through a physical interaction between RARα and Nrf2, an association that is greatly promoted by retinoic acid and prevents Nrf2 from binding to the ARE. Antagonism of Nrf2 by retinoids or BTB09463 increases the sensitivity of MCF7 cells [with Nrf2 that is negatively controlled by Keap1] as well as A549 cells [with Nrf2 that is not controlled by Keap1] to the cytotoxic effects of Bleomycin and Carmustine.

Our invention also includes the generation and validation of the MCF7-derived reporter cell line, called AREc32, which contains a concatenated synthetic ARE-luciferase reporter gene that is highly responsive to tBHQ and Sul. The use of AREc32 cells was used to screen a 6000 chemical library from which BTB09463 was identified as an inhibitor of ARE-luciferase induction by tBHQ. Separately, the AREc32 cells were also used to identify retinoids as inhibitors or ARE-luciferase induction by tBHQ.

TABLE 1
Sequence of inserts in the pGL3 promoter vector.
The minimal enhancer sequence
5′ A/G TGACnnnGC A/G-3′, present as either a
single or multiple copies within the inserts
for the various reporter constructs is shown
underlined.

| Plasmid | Sequence of insert (5′→3′) |
|---|---|
| PGL-1xARE | 5′-CCCGTGACAAAGCACCC-3′ |
| PGL-2xARE | 5′-GTGACAAAGCACCCGTGACAAAGCA-3′ |
| PGL-4xARE | 5′GTGACAAAGCACCCGTGACAAAGCACCCGTGA |
| | CAAAGCACCCGTGACAAAGCA-3′ |
| PGL-6xARE | 5′GTGACAAAGCACCCGTGACAAAGCACCCGTGA |
| | CAAAGCACCCGTGACAAAGCACCCGTGACAAAGC |
| | ACCCGTGACAAAGCA-3′ |
| PGL-8xARE | 5′GTGACAAAGCACCCGTGACAAAGCACCCGTGA |
| | CAAAGCACCCGTGACAAAGCACCCGTGACAAAGC |
| | ACCCGTGACAAAGCACCCGTGACAAAGCACCCGT |
| | GACAAAGCA-3′ |
| PGL-GSTA2.41 bp- | 5′-GAGCTTGGAAATGGCATTGCTAATGGTGACA |
| ARE | AAGCAACTTTG-3′ |

TABLE 2
Identification of MCF7 cells for optimal use of ARE reporter system

| | Relative luciferase | Relative luciferase | |
|---|---|---|---|
| | activity | activity | Ratio |
| Cell line | (DMSO treated) | (tBHQ treated) | (tBHQ/DMSO) |
| HepG2 | 1.0 ± 0.3 | 2.8 ± 0.9 | 2.8 ± 0.9 |
| MCF7 | 43.8 ± 3.5 | 2276.1 ± 521.1 | 52.0 ± 11.9 |
| CHO | 426.1 ± 64.7 | 1171.6 ± 8.8 | 2.7 ± 0.1 |
| Hepa1 | 39.2 ± 1.4 | 140.7 ± 19.6 | 3.6 ± 0.5 |

MCF7, HepG2, CHO and Hepa1 cells were seeded at 1 × 10⁵ cells/well in 24-well plates, transfected with pGL-GSTA2.41bp-ARE construct. The plasmid pRL-TK was used as internal control in each transfection. The cells were use treated with 50 μM tBHQ and luciferase reporter activity determined as detailed in the Materials and Methods. For control experiments, the same volume of DMSO was added to the medium. The value of relative luciferase activity of HepG2 cells treated with DMSO was set at 1. This represents the results of three separate experiments. Each treatment in each experiment has at least three replicates.

TABLE 3
Inducers of luciferase activity in AREc32 cells.

| Compound | CD* (μM) |
|---|---|
| tBHQ | 1 |
| SUL | 2 |
| Acrolein | 2 |
| Ethoxyquin | 5 |
| BHA | 20 |
| I3C | 20 |
| PDTC | 20 |
| MMS | 100 |
| 7-ethoxycoumarin | 100 |
| H₂O₂ | 300 |

Cells were seeded in a 96-well plate at 1.2 × 10⁴ cells/well in the growth medium. After 24 h recovery, the culture medium was replaced with fresh DMEM supplemented with antibiotics containing various concentrations of the compounds listed below. The cells were then incubated for 24 h, and assayed for luciferase activity as detailed in the Materials and Methods. The value of luciferase activity of cells treated with DMSO (0.1% v/v) was set at 1. The results presented represent results from three separate experiments. Each treatment in each experiment has at least three replicates.
*CD, concentration of inducting agent that doubled luciferase reporter activity.

TABLE 4
Effect of the treating AREc32 cells with
anticancer drugs and their metabolites.

| Type of | Drugs and | | | |
|---|---|---|---|---|
| modulation | metabolites | Fold increase^{a} | Conc. | |
| Inactive | Doxorubicin | 1.0 ± 0.04 | 1.0 | μg/ml |
| | Epirubicin | 1.1 ± 0.03 | 1.0 | μg/ml |
| | Cyclophosphamide | 1.0 ± 0.05 | 100 | μM |
| | Methotrexate | 1.1 ± 0.06 | 10 | μM |
| | Paclitaxol | 1.1 ± 0.05 | 5 | nM |
| | Thiotepa | 1.1 ± 0.1 | 20 | μM |
| Weak inducers | Cisplatin* | 1.3 ± 0.06 | 10 | μM |
| | Mephalan* | 1.3 ± 0.06 | 20 | μM |
| | Etopside* | 1.3 ± 0.07 | 10 | μM |
| | Chlorambucil* | 1.8 ± 0.19 | 100 | μM |
| | Mitozantrone* | 2.1 ± 0.08 | 1 | μM |
| | BCNU* | 4.1 ± 0.15 | 100 | μM |
| Strong inducer | Acrolein | 27 ± 2.5 | 10 | μM |

Treatment was 24 h as detailed in Materials and Methods. For control cells, the same volume of 0.1% (v/v) of vehicle was added to the medium. The significant of the differences between luciferase activity from cultures exposed to the anticancer agents and cultures treated with the DMSO was assessed by unpaired student's t-test. This represents the results of three separate experiments.
*p < 0.05.
^{a}Data expressed as mean-fold increase relative to control value ± S.D.

TABLE 5
Sensitization of tumour cells to the cytotoxic effects
of anticancer drugs by BTB09463 or retinoids

| Cell line | Treatment | IC₅₀ μM |
|---|---|---|
| MCF7 | BTB09463 | 28 |
| | Carmustine | 291 |
| | Carmustine & BTB09463 (10 μM) | 191 |
| | Bleomycin | 660 |
| | Bleomycin & BTB09463 (5 μM) | 250 |
| | Bleomycin & all-trans Retinoic acid (0.5 μM) | 250 |
| | Bleomycin & all-trans Retinal (0.5 μM) | 127 |
| | Bleomycin & Retinyl acetate (0.5 μM) | 111 |
| A549 | BTB09463 | 52 |
| | Carmustine | >1500 |
| | Carmustine & BTB09463 (20 μM) | 400 |
| | Carmustine & all-trans Retinoic acid (0.5 μM) | 250 |
| | Bleomycin | 55 |
| | Bleomycin & BTB09463 (20 μM) | 5.7 |
| | Bleomycin & all-trans Retinoic acid (0.5 μM) | 19 |

### REFERENCES

1. Aoki, Y., Sato, H., Nishimura, N., Takahashi, S., Itoh, K. and Yamamoto, M. (2001) Toxicol. Appl. Pharmacol. 173, 154-160.
2. Bonnesen, C., Eggleston, I. M. and Hayes, J. D. (2001) Cancer Res. 61, 6120-6130.
3. Chan, K. and Kan, Y. W. (1999) Proc. Natl. Acad. Sci. USA 96, 12731-12736.
4. Chanas, S. A., Jiang, Q., McMahon, M., McWalter, G. K., McLellan, L. I. Elcombe, C. R., Henderson, C. J., Wolf, C. R., Moffat, G. J., Itoh, K., Yamamoto, M. and Hayes, J. D. (2002) Biochem. J. 365, 405-16.
5. Cho, H. Y., Jedlicka, A. E., Reddy, S. P., Kensler, T. W., Yamamoto, M., Zhang, L. Y. and Kleeberger, S. R. (2002) Am. J. Respir. Cell. Mol. Biol. 26, 175-182.
6. Cullinan, S. B., Gordan, J. D., Jin, J., Harper, J. W. and Diehl, J. A. (2004) Mol. Cell. Biol. 24, 8477-8486.
7. Deng, W. G., Zhu, Y., Montero, A. and Wu, K. K. (2003) Anal. Biochem. 323, 12-18
8. Devling, T. W., Lindsay, C. D., McLellan, L. I. McMahon, M. and Hayes, J. D. (2005) Proc. Natl. Acad. Sci. USA 102, 7280-7285A.
9. Enomoto, A., Itoh, K., Nagayoshi, E., Haruta, J., Kimura, T., O'Connor, T., Harada, T. and Yamamoto, M. (2001) Toxicol. Sci. 59, 169-177.
10. Erickson, A. M., Nevarea, Z., Gipp, J. J. and Mulcahy, R. T. (2002) J. Biol. Chem. 277, 30730-30737.
11. Friling, R. S., Bergelson, S. and Daniel, V. (1992) Proc. Natl. Acad. Sci. USA 89, 668-672.
12. Furukawa, M. and Xiong, Y. (2005) Mol. Cell. Biol. 25,162-171.
13. Hayes, J. D. and McMahon, M. (2001) Cancer Lett. 174, 103-113.
14. Hayes, J. D. and Pulford, D. J. (1995) Crit. Rev. Biochem. Mol. Biol. 30, 445-600.
15. Hayes, J. D. and Wolf, C. R. (1990) Biochem. J. 272, 281-295.
16. Hayes, J. D., Chanas, S. A., Henderson, C. J., McMahon, M., Sun, C., Moffat, G. J., Wolf, C. R. and Yamamoto, M. (2000) Biochem. Soc. Trans. 28, 33-41
17. Hong, F., Freeman, M. L. and Liebler, D. C. (2005) Chem. Res. Toxicol. 18, 1917-1926.
18. Iida, K., Itoh, K., Kumagai, Y., Oyasu, R., Hattori, K., Kawai, K., Shimazui, T., Akaza, H. and Yamamoto, M. (2004) Cancer Res. 64, 6424-6431.
19. Ikeda, H., Serria, M. S., Kakizaki, I., Hatayama, I., Satoh, K., Tsuchida, S., Muramatsu, M., Nishi, S. and Sakai, M. (2002) Biochem. J. 364, 563-570.
20. Itoh, K., Chiba, T., Takahashi, S., Ishii, T., Igarashi, K., Katoh, Y., Oyake, T., Hayashi, N., Satoh, K., Hatayama, I., Yamamoto, M. and Nabeshima, Y. (1997) Biochem. Biophys. Res. Commun. 236, 313-322.
21. Itoh, K., Wakabayashi, N., Katoh, Y., Ishii, T., Igarashi, K., Engel, J. D. and Yamamoto, M. (1999) Genes Dev. 13, 76-86.18. Jowsey, I. R., Jiang, Q., Itoh, K., Yamamoto, M. and Hayes, J. D. (2003) Mol. Pharmacol. 64, 1018-1028.
22. Kang, M. I., Kobayashi, A., Wakabayashi, N., Kim, S. G. and Yamamoto, M. (2004) Proc. Natl. Acad. Sci. USA 101, 2046-2051
23. Kepa, J. K. and Ross, D. (2003) Biochem. Biophys. Res. Commun. 311, 446-453.
24. Kawabe, T., Chen, Z. S., Wada, M., Uchiumi, T., Ono, M., Akiyama, S. and Kuwano, M. (1999) FEBS Lett. 456, 327-331
25. Kelly, V. P., Ellis, E. M., Manson, M. M., Chanas, S. A., Moffat, G. J., McLeod, R., Judah, D. J., Neal, G. E. and Hayes J D (2000) Cancer Res. 60: 957-969
26. Kizaki, M., Sakashita, A., Karmakar, A., Lin, C. W. and Koeffler, H. P. (1993) Blood 82, 1142-50.
27. Kobayashi, M. and Yamamoto, M. (2005) Antioxid. Redox Signal 7, 385-394.
28. Kobayashi, A., Kang, M. I., Okawa, H., Ohtsuji, M., Zenke, Y., Chiba, T., Igarashi, K. and Yamamoto, M. (2004) Mol. Cell. Biol. 24, 7130-7139.
29. Lee, J. M., Calkins, M. J., Chan, K., Kan, Y. W. and Johnson, J. A. (2003) J. Biol. Chem. 278, 12029-12038.
30. McLellan, L. I. and Wolf, C. R. (1999) Drug Resist. Update 2, 153-164.
31. McMahon, M., Itoh, K., Yamamoto, M., Chanas, S. A., Henderson, C. J., McLellan, L. I., Wolf, C. R., Cavin, C. and Hayes, J. D. (2001) Cancer Res. 61, 3299-3307.
32. McMahon, M., Itoh, K., Yamamoto, M. and Hayes, J. D. (2003) J. Biol. Chem. 278, 21592-21600.
33. Malkinson, A. M., Siegel, D., Forrest, G. L., Gazdar, A. F., Oie, H. K., Chan, D. C., Bunn, P. A., Mabry, M., Dykes, D. J., Harrison, S. D. and et al. (1992) Cancer Res. 52, 4752-4757.
34. Moffat, G. J., McLaren, A. W. and Wolf, C. R. (1997) Biochem. J. 324, 91-95.
35. Motohashi, H., O'Connor, T., Katsuoka, F., Engel, J. and Yamamoto, M. (2002) Gene 294, 1-12
36. Mulcahy, R. T., Bailey, H. H. and Gipp, J. J. (1995) Cancer Res 55, 4771-5.
37. Mulcahy, R. T., Wartman, M. A., Bailey, H. H. and Gipp, J. J. (1997) J. Biol. Chem. 272, 7445-7454.
38. Nioi, P. and Hayes, J. D. (2004) Mutat. Res. 555, 149-171.
39. Nioi, P., McMahon, M., Itoh, K., Yamamoto, M. and Hayes, J. D. (2003) Biochem. J. 374, 337-348.
40. Nguyen, T., Rushmore, T. H. and Pickett, C. B. (1994) J. Biol. Chem. 269, 13656-13662
41. O'Connor, T., Ireland, L. S., Harrison, D. J. and Hayes, J. D. (1999) Biochem. J. 343, 487-504
42. Ogretmen, B., Bahadori, H. R., McCauley, M. D., Boylan, A., Green, M. R. and Safa, A. R. (1998) Int. J. Cancer 75, 757-761.
43. Ramos-Gomez, M., Kwak, M. K., Dolan, P. M., Itoh, K., Yamamoto, M., Talalay, P. and Kensler, T. W. (2001) Proc. Natl. Acad. Sci. USA 98, 3410-3415.
44. Rushmore, T. H., Morton, M. R. and Pickett, C. B. (1991) J. Biol. Chem. 266, 11632-11639
45. Sasaki, H., Sato, H., Kuriyama-Matsumura, K., Sato, K., Maebara, K., Wang, H., Tamba, M., Itoh, K., Yamamoto, M. and Bannai, S. (2002) J. Biol. Chem. 277, 44765-44771
46. Schlager, J. J. and Powis, G. (1990) Int. J. Cancer 45, 403-409. Smitherman, P. K., Townsend, A. J., Kute, T. E. and Morrow, C. S. (2004) J. Pharmacol. Exp. Ther. 308, 260-267
47. Smitskamp-Wilms, E., Giaccone, G., Pinedo, H. M., van der Laan, B. F. and Peters, G. J. (1995) Br. J. Cancer 72, 917-921.
48. Tew, K. D. (1994) Cancer Res 54, 4313-4320.
49. Townsend, D. M. and Tew, K. D. (2003) Oncogene 22, 7369-7375.
50. Townsend, D. M., Tew, K. D. and Tapiero, H. (2003) Biomed. Pharmacother. 57, 145-155.
51. Vlaming, M. L., Mohrmann, K., Wagenaar, E., de Waart, D. R., Elferink, R. P., Lagas, J. S., van Tellingen, O., Vainchtein, L. D., Rosing, H., Beijnen, J. H., Schellens, J. H. and Schinkel, A. H. (2006) J. Pharmacol. Exp. Ther. 318, 319-327
52. Wakabayashi, N., Itoh, K., Wakabayashi, J., Motohashi, H., Noda, S., Takahashi, S., Imakado, S., Kotsuji, T., Otsuka, F., Roop, D. R., Harada, T., Engel, J. D. and Yamamoto, M. (2003) Nat. Genet. 35, 238-245.
53. Wang, X. J., Chamberlain, M., Vassieva, O., Henderson, C. J. and Wolf, C. R. (2005) Biochem. J. 388, 857-867
Wang, X. J., Hayes, J. D. and Wolf, C. R. (2006) submitted for publication
54. Wasserman, W. W. and Fahl, W. E. (1997) Proc. Natl. Acad. Sci. USA 94, 5361-5366
55. Waxman, D. J. (1990) Cancer Res. 50, 6449-6454.
56. Wong, G. H. (1995) Biochim. Biophys. Acta 1271, 205-209.
57. Yao, K. S., Godwin, A. K., Johnson, S. W., Ozols, R. F., O'Dwyer, P. J. and Hamilton, T. C. (1995) Cancer Res. 55, 4367-4374.
58. Zhang, D. D., Lo, S. C., Cross, J. V., Templeton, D. J. and Hannink, M. (2004) Mol. Cell. Biol. 24, 10941-10953.
59. Zhang, D. D., Lo, S. C., Sun, Z., Habib, G. M., Lieberman, M. W. and Hannink, M. (2005) J. Biol. Chem. 280, 30091-30099.
60. Zhu, M. and Fahl, W. E. (2000) Anal. Biochem. 287, 210-217

1-41. (canceled)

42. A method of treating a subject in need thereof comprising administering an agent, which is capable of down-regulating Nrf2 gene activity.

43. The method according to claim 42 wherein the agent down-regulates transactivation of gene expression by Nrf2.

44. The method according to claim 43 wherein the gene(s), the expression of which are down-regulated by reduced transactivation by Nrf2, is/are a gene associated with the ARE gene pathway.

45. The method according to claim 42 wherein the agent is for treating diseases associated with abnormal cell proliferation.

46. The method according to claim 42 wherein the agent is intended to be administered alone.

47. The method according to claim 42 further comprises administering another agent selected from the group consisting of a cytotoxic agent, alkylating agent, redox cycling agent, thiol-active chemical, inhibitor of GSH synthesis, or a pro-apoptotic agent.

48. The method according to claim 42 wherein the subject to be treated is also to be treated by UV ionising radiation or photodynamic therapy.

49. The method according to claim 44 wherein the disease to be treated is a cancer or psoriasis.

50. A method of treating a disease associated with abnormal cell proliferation comprising administering to a subject in need thereof a retinoid or compound according to Formula (I) wherein the retinoid sensitises an abnormally proliferating cell in a host by way of down-regulating ARE-driven gene expression: 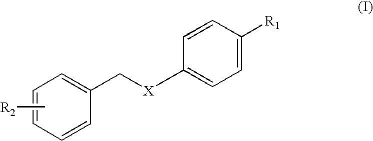
wherein X is C, O, N or S; R₁, is C₁-C₄ alkyl, C₁-C₄(OH), COOH, C(═CH₂)CH₃, C(═O)CH₃, CH(CH₃)₂, C(CH₃)₃; and R₂ is independently selected from, at each available position, H, halo, C₁-C₄ alkyl, OH or NH₂.

51. The method according to claim 50 wherein the retinoid down-regulates Nrf2 gene activity thereby increasing servitivity of an antiproliferative agent against which Nrf2 would confer a degree of protection.

52. The method according to claim 50 further comprising administering a further agent which is capable of inducing cell death of a cell sensitised by the retinoid.

53. The method according to claim 52 wherein the further agent is a chemotherapeutic agent such as an alkylating agent or a redox cycling agent.

54. The method according to claim 50 wherein the retinoid down-regulates the transactivation of gene expression by Nrf2.

55. The method according to claim 50 wherein the retinoid is all trans-retinoic acid, 9-cis retinoic acid, 13-cis retinoic acid, retinal or retinol.

56. The method according to claim 50 wherein the retinoid is

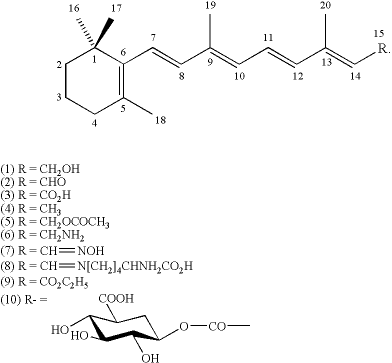

57. A pharmaceutical composition comprising, or consisting essentially of, as active ingredients, an agent capable of down-regulating Nrf2 activity, such as a retinoid and a chemotherapeutic agent.

58. The pharmaceutical composition according to claim 57 wherein the chemotherapeutic agent is an alkylating agent or a redox cycling compound.

59. The pharmaceutical composition according to claim 58 wherein the chemotherapeutic agent is cisplatin, melphalan, chlorambucil, mitrozantrone, BCNU, thistepa, doxorubicin or bleomycin.

60. The pharmaceutical composition according to claim 57 further comprising a redox controlling agent, such as BSO, that inhibits GSH production or thioredoxin production.

61. A method of screening for agents which directly or indirectly down-regulate induction of ARE-driven gene expression, for use in sensitising cells to cytotoxicity or apoptosis, comprising the steps of:
a) providing in vitro a cell which is capable of driving an antioxidant response, wherein the cell comprises an ARE-reporter gene construct comprising a reporter gene located downstream and controlled by multiple concatenated ARE sequences;
b) contacting a test agent to be screened with said cell; and
c) detecting whether or not said agent is capable of decreasing induction or decreasing expression of the reporter gene, in comparison to a cell to which the test agent has not been added.

62. The method according to claim 61 for identifying agents which may be of use in treating diseases associated with abnormal cell proliferation.

63. The method according to claim 62 wherein the disease is cancer or psoriasis.

64. The method according to claim 61 wherein the test agent is also tested for its ability to inhibit Nrf2 activity.

65. The method according to claim 61 wherein the ARE sequence used is from the rat GSTA2 and/or mouse gsta1 genes.

66. The method according to claim 61 wherein the cell is a tumour cell.

67. The method according to any claim 61 wherein the cell is a mammalian cell which is capable of driving an antioxidant response.

68. The method according to claim 61 wherein the cell is a MCF7 cell.

69. The method according to claim 61 wherein induction of the reporter gene is enhanced by addition of an activating agent.

70. The method according to claim 69 wherein the activating agent is a quinone such as tBHQ, and isothiocyamate, such as sulforaphane, a α, β-unsaturated carbonyl, such as diethyl maleate or a flavonoid, such as β-naphthoflavone, or an epithioalkane, such as 1-cyano-2,3-epithiopropane, or a di-mercaptan, such as lipoic acid.

71. The method according to claim 64 wherein Nrf2 is activated by down regulating expression of Keap1 using antisense or RNAi techniques, or within a cell containing a mutant Keap1 gene.

72. The method according to claim 64 wherein Nrf2 is activated by down-regulating the expression of negatively-acting competing transcription factors such as Bach1, Bach2, cFos and small Maf.

73. The method according claim 61 wherein the reporter gene is GFP and related fluorescent proteins, luciferase, β-galactosidase, or chloramphenicol acetyl transferase, alkaline phosophatase or any assayable hormone or enzyme.

74. The method according to claim 61 wherein detection of a product of the reporter gene is carried out by a colorimetric, fluorimetric, luminescent, radioimmuno or immunological assay.

75. The method according to claim 61 wherein a comparison or control experiment is carried out to ascertain a level or degree of reporter activity, in the absence of the test agent.

76. A cell for use in screening agents for an effect on ARE-driven gene expression, wherein the cell is a human mammary MCF7 cell containing an ARE reporter construct that comprises a reporter gene downstream of multiple concatenated copies of the ARE sequence from the rat GSTA2 and/or mouse gsta1 genes.

77. The cell according to claim 76 wherein the reporter gene is a luciferase gene.

78. The cell according to claim 77 wherein the multiple concatenated ARE sequences are located head-to-tail, in series, upstream of the reporter gene.

79. The cell according to claim 77 wherein the numbers of copies are 6-8.


SEQUENCE LISTING (11 sequences)

SEQ ID NO: 1 - DNA, 11 bp, Rattus rattus
  misc_feature (1)..(1): n is A or G
ntgacnnngcn

SEQ ID NO: 2 - DNA, 11 bp, Rattus rattus
gtgacaaagca

SEQ ID NO: 3 - DNA, 42 bp, Rattus rattus
gagcttggaaatggcattgctaatggtgacaaagcaactttg

SEQ ID NO: 4 - DNA, 40 bp, Mus musculus
tagcttggaaatgacattgctaatggtgacaaagcaactg

SEQ ID NO: 5 - DNA, 19 bp, Homo sapiens
gcattggagtgtcagtatg

SEQ ID NO: 6 - DNA, 22 bp, Artificial Sequence
  Minimal enhancer sequence
agagtggtcactaccccctctg

SEQ ID NO: 7 - DNA, 17 bp, Artificial Sequence
  Minimal enhancer sequence
cccgtgacaaagcaccc

SEQ ID NO: 8 - DNA, 25 bp, Artificial Sequence
  Minimal enhancer sequence
gtgacaaagcacccgtgacaaagca

SEQ ID NO: 9 - DNA, 53 bp, Artificial Sequence
  Minimal enhancer sequence
gtgacaaagcacccgtgacaaagcacccgtgacaaagcacccgtgacaaagca

SEQ ID NO: 10 - DNA, 81 bp, Artificial Sequence
  Minimal enhancer sequence
gtgacaaagcacccgtgacaaagcacccgtgacaaagcacccgtgacaaagcacccgtgacaaagcacccgtgacaaagca

SEQ ID NO: 11 - DNA, 109 bp, Artificial Sequence
  Minimal enhancer sequence
gtgacaaagcacccgtgacaaagcacccgtgacaaagcacccgtgacaaagcacccgtgacaaagcacccgtgacaaagcacccgtgacaaagcacccgtgacaaagca